(19) United States
(12) Patent Application Publication
(10) Pub. No.: US 2023/0057308 A1
(43) Pub. Date: Feb. 23, 2023

(54) PREDICTION OF BIOLOGICAL ROLE OF TISSUE RECEPTORS

(71) Applicant: CARMEL HAIFA UNIVERSITY ECONOMIC CORPORATION LTD., Haifa (IL)

(72) Inventor: Judith SOMEKH, Kerem-Maharal (IL)

(21) Appl. No.: 17/980,358

(22) Filed: Nov. 3, 2022

Related U.S. Application Data
(63) Continuation of application No. PCT/IL2021/050509, filed on May 4, 2021.
(60) Provisional application No. 63/019,523, filed on May 4, 2020.

(51) Int. Cl. G06N 20/10; G16B 5/00; G16B 30/00; G06K 9/62

(52) U.S. Cl. CPC G06N 20/10; G06K 9/6259; G06K 9/6276; G16B 5/00; G16B 30/00

(57) ABSTRACT

Computerized methods to predict or determine if a receptor is associated with a biological process in a target tissue are provided. Methods of training and using a machine learning model to predict or determine if a receptor is associated with a biological process in a target tissue as well as systems and computer program product to do same are also provided.

### CROSS REFERENCE TO RELATED APPLICATIONS

This application is a Bypass Continuation of PCT Patent Application No. PCT/IL2021/050509 having International filing date of May 4, 2021, which claims the benefit of priority of U.S. Provisional Pat. Application No. 63/019,523 titled “PREDICTION OF BIOLOGICAL ROLE OF TISSUE RECEPTORS”, filed May 4, 2020, the contents of which are all incorporated herein by reference in their entirety.

### FIELD OF INVENTION

The present invention is in the field of machine learning for biological function analysis.

### BACKGROUND OF THE INVENTION

The human system, as any other biological system, always aiming to achieve a state of homeostasis, responds to different conditions through activating feedback control loops between its sub-systems, organs and tissues. For example, to ensure whole organism survival, the endocrine system preserves long feedback loops of ligands secretion and receptors binding to maintain glucose or energetic balance. Ligand-receptor secretion and binding are accomplished by molecules, i.e., ligands, secreted into the blood stream from source organs that bind to receptors located on both the cell surface and within the cells of target organs. This complex network of whole-body ligand-receptor interactions serve as the information transducer of these feedback loops. Understanding these receptor roles is pivotal in the field of modern medicine. Receptor dysregulation underlies the etiology of many human diseases (e.g., diabetes) and prescription drugs are designed to affect the regulation of receptors and produce therapeutic changes in the function of related biological systems. Moreover, receptors serve as targets for virus invasion of cells. Currently, countless efforts are being made to develop drugs that can disrupt this interaction between the ligand and its receptors. Albeit years of research, the present-day understanding of the tissue-specific functions of many receptors and their ligand intercellular signalling networks is still incomplete. Developing drugs continues to be a challenge, as advances in scientific knowledge of receptors has been relatively slow, being based on laborious experimentation that typically precedes testing one or two receptors at a time in one or two tissues.

The advent of ultrahigh-throughput sequencing technologies and algorithmic advancements now enable systematically and simultaneously investigation of hundreds of genes coded to receptors. Recent computational work has defined cross-tissue expression of ligand-receptor pairs by merely measuring the expression levels of ligands and receptors across 144 cell types. A common task of analysis of gene expression data is to detect gene-gene co-expression networks. These gene co-expression networks are based on the “guilt by association” concept that is related to the fact that functionally related genes are often co-expressed. Such networks are used to identify the functional roles of genes whose function is unknown by relating their co-expression networks to known biological processes. Understanding hormonal signaling pathways would be tremendously significant in many areas of systems biology, drug discovery, and modern medicine. However, to date, this communication process is only partially understood.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

The present invention provides methods of training a machine learning model to predict receptors associated with a biological process in a target tissue. Methods of determining receptors that are associated with a biological process or associated with the biological process in a target tissue are also provided, as are systems and computer program products for doing same.

According to a fist aspect, there is provided a method comprising:

training a machine learning model to predict receptors associated with a biological process in a target tissue, on a training set, the method comprising:
receiving a first list of receptors known to be associated with the biological process and expressed in the target tissue and a second list of receptors known to be expressed in the target tissue and not associated with the biological process;
receiving a dataset comprising expression profiles in the target tissue for genes encoding proteins, wherein the proteins include receptors of the first list and receptors of the second list;
applying, to the dataset, co-expression network analysis to group the genes into clusters, based on a co-expression relationship between the genes in the target tissue;
applying to the clusters, a pathways enrichment analysis to assign enrichment scores to the clusters for each pathway of the enrichment analysis, wherein each pathway is a pathway of a specific biological process;
labeling receptors from the first list and receptors from the second list with labels comprising the enrichment score for each pathway assigned to a cluster containing the gene encoding the receptor;
generating an annotated training set comprising receptors from the first list and receptors from the second list and corresponding labels; and training the machine learning model on the annotated training set to produce a trained machine learning model.

According to another aspect, there is provided a method comprising:
receiving, as input, a dataset comprising gene expression profiles with respect to a plurality of genes associated with a corresponding plurality of tissues, wherein at least one of the genes encode a receptor;
applying, to the dataset, gene co-expression network analysis to group the genes into tissue-specific clusters, based on a co-expression relationship between the genes in tissues of the plurality of tissues;
applying, to the tissue-specific clusters, a pathways enrichment analysis to assign an enrichment score to the tissue-specific clusters, wherein at least one of the pathways is a pathway of a specific biological process; and
identifying a gene encoding a receptor included in more than one cluster having an enrichment score for a pathway of the specific biological process above a predetermined threshold as a receptor which is associated with the specified biological process.

According to another aspect, there is provided a method of determining if a receptor is associated with a biological process in a target tissue, the method comprising:
receiving, as input, a receptor of unkonwn association with the biological process in the target tissue, and enrichment scores for pathways assigned to a cluster of genes containing a gene encoding the receptor of unknown association, wherein the cluster is generated by gene co-expression network analysis of expression profiles in the target tissue of a set of genes which includes the gene encoding the receptor of unknown association; and
applying a trained machine learning model to the input to determine if the receptor of unknown association is associated with the biological process in the target tissue, wherein the trained machine learning model has been trained on a training set comprising a first list of receptors known to be associated with the biological process and expressed in the target tissue labeled with labels comprising enrichment scores for pathways assigned to a cluster of genes containing a gene encoding the receptors of the first list, and a second list of receptors known to be expressed in the target tissue and not associated with the biological process labeled with labels comprising enrichment scores for pathways assigned to a cluster of genes containing a gene encoding receptors of the second list;
thereby determining if a receptor is associated with a biological process in a target tissue.

According to another aspect, there is provided a system comprising:
at least one hardware processor; and
a non-transitory computer-readable storage medium having stored thereon program code, the program code executable by the at least one hardware processor to perform a method of the invention.

According to another aspect, there is provided a computer program product comprising a non-transitory computer-readable storage medium having program code embodied therewith, the program code executable by at least one hardware processor to perform a method of the invention.

According to some embodiments, the receptors are cell surface receptors, internal receptors within a cell or a combination thereof.

According to some embodiments, the expression profiles are mRNA expression profiles.

According to some embodiments, the gene co-expression network analysis comprises employing a Weighted Gene Co-Expression Network Analysis (WGCNA) algorithm.

According to some embodiments, the co-expression relationship is determined from the expression profiles.

According to some embodiments, the receptors known to be associated with the biological process have been experimentally confirmed to be associated with the biological process.

According to some embodiments, the receptors known to not be associated with the biological process are determined using a Positive unlabeled (PU) support vector machines (SVM) bagging algorithm.

According to some embodiments, the dataset comprises expression profiles for all genes expressed in the target tissue.

According to some embodiments, the pathways enrichment analysis comprises KEGG pathway analysis.

According to some embodiments, the enrichment score is an enrichment score for an entire cluster and not for an individual gene.

According to some embodiments, the machine learning model is selected from a SVM classifier and a k-nearest neighbor (k-NN) classifier.

According to some embodiments, the machine learning model is a k-NN classifier.

According to some embodiments, the method of the invention further comprises:
at an inference stage, receiving, as input, a receptor absent from the annotated training set, and enrichment scores for pathways assigned to a cluster of genes containing a gene encoding the receptor absent from the annotated training set, wherein the cluster of genes containing the gene encoding the receptor absent from the annotated training set is generated by co-expression network analysis of expression profiles of genes in the target tissue; and
applying the trained machine learning model to the input to identify a receptor associated with the biological process in the target tissue.

According to some embodiments, the receptor absent from the annotated training set is a receptor of unknown association with the biological process in the target tissue.

According to some embodiments, the receptor absent from the annotated training set is a receptor absent from the first list and the second list.

According to some embodiments, the enrichment scores for pathways assigned to a cluster of genes containing the gene that encodes the receptor absent from the annotated training set are generated by a method comprising: receiving a dataset comprising expression profiles in the target tissue for genes encoding proteins, wherein the proteins include the receptor absent from the annotated training set, applying, to the dataset, co-expression network analysis to group the genes into clusters, based on a co-expression relationship between the genes in the target tissue; and applying to the clusters, a pathways enrichment analysis to assign enrichment scores to the clusters for each pathway of the enrichment analysis, wherein each pathway is a pathway of a specific biological process.

According to some embodiments, the receptor is selected from a cell surface receptor and an internal receptor within a cell.

According to some embodiments, the identifying comprises identifying genes encoding receptors included in at least five clusters having an enrichment score for a pathway of the specific biological process above a predetermined threshold.

According to some embodiments, the identifying further comprises identifying genes encoding receptors with correlation to an eigengene of the cluster above a predetermined threshold.

According to some embodiments, the identifying comprises applying a machine learning model trained on receptors associated with the biological process and receptors not associated with the biological process.

According to some embodiments, the machine learning model is trained by a method of the invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Exemplary embodiments are illustrated in referenced figures. Dimensions of components and features shown in the figures are generally chosen for convenience and clarity of presentation and are not necessarily shown to scale. The figures are listed below.

FIGS. 1A-C: A schematic illustration of an overview of a method for computerized classification and prediction of tissue-specific roles of receptors. (1A) An annotated list of metabolic and non-metabolic receptors in adipose is generated. (1B) Co-expression network analysis is used to generate gene modules (clusters) in subcutaneous adipose, which is followed by pathways enrichment analysis. Enrichment scores are used to train machine learning classifiers. (1C) Performance of the classifiers is validated and evaluated.

FIGS. 2A-C: Pathway enrichment analysis of the labeled metabolic receptors related modules in subcutaneous adipose. (2A) A heatmap of log-transformed p-values (adjusted for multiple correction) of the KEGG pathways enrichment analysis is presented. Enriched pathways for the metabolic and non-metabolic receptors are used for training. It can be seen that the metabolic receptors (highlighted in green in the annotated columns) form a metabolic cluster (highlighted in the annotation rows to the right in turquois and corresponding to the KEGG metabolism hierarchical classification) (2B) A heatmap focusing on the metabolic receptors enriched pathways which shows that they are highly enriched with various metabolic pathways. The rows represent the KEGG pathways, and the columns, the receptors (e.g., insulin receptor (INSR)). Multiple metabolic receptors are included in Module 1 in subcutaneous adipose, which is enriched with metabolic pathways. (2C) Predicted metabolic receptors in adipose and similar tissues. Key driver receptors of the Adipose-Subcutaneous metabolic module (ME1). The strength of the color represents the module’s receptor key drivers, i.e., the strength of the correlation between the receptor and the eigengene of the metabolic module (i.e., the first principal component of the module). The module eigengene corresponds to the first principal component of a given module and is considered the most representative gene expression in a module. It can be seen that among others, the known growth hormone receptor (GHR) and insulin receptor (INSR) are predicted to have a metabolic function and are correlated with the metabolic module (r²=0.8 and r²=0.42, respectively). The edge width represents the weight of the receptor co-expression values generated by the WGCNA algorithm, based on correlation values and topological similarity (TOM, adjacent nodes that have similar neighbors) between the receptors. Receptors that are also predicted to have a metabolic function in Adipose-Visceral are highlighted with yellow circles.

FIG. 3: Clustering analysis showing metabolic tissues and their clustering, in accordance with some embodiments of the present invention.

FIG. 4: Bar graph showing tissues that include “pure metabolic” receptors and their count per tissue, in accordance with some embodiments of the present invention; and

FIG. 5: Bar graph presenting the strongest metabolic receptors that exhibit metabolic roles in most of the examined tissues, in accordance with some embodiments of the present invention.

FIGS. 6A-C: (6A) Table listing known hormones and their receptors derived from Antonescu, et al., 2014 “Reciprocal regulation of endocytosis and metabolism”; Vijayakumar, et al., 2011, “The intricate role of growth hormone in metabolism”; and Luo, et al., 2016, “Adipose tissue in control of metabolism”. All of which are hereby incorporated by reference in their entirety. “Relevant” columns mean that the receptor is present in the GTEx database or in the tested receptors list. A total of 17 metabolic receptors are valid (expressed or included in modules of subcutaneous adipose) to be tested in subcutaneous adipose. (6B) Table listing positive receptors inferred by bagging. (6C) Table of enrichment results of 55 negatively labeled receptors (negative rate > 0.8) inferred by PU SVM bagging algorithm.

FIG. 7: A schematic illustration an overview of a method for computerized classification and prediction of tissue-specific roles of receptors, in accordance with some embodiments of the present invention.

FIG. 8: A flowchart detailing the functional steps in a process for computerized classification and prediction of tissue-specific roles of receptors, in accordance with some embodiments of the present invention.

FIG. 9: A heatmap of log-transformed p-values (adjusted for multiple correction) of the enrichment analysis using the KEGG pathways of metabolic pathways of tissue-specific modules.

FIG. 10: A heatmap showing four tissues that are closely clustered with Adipose-Subcutaneous and their common metabolic receptors (presented receptors are common for at least two tissues). In the heatmap, each column represents a tissue, and each row represents a receptor. Receptors were annotated and colored in the heatmap as genetic-metabolic (light orange), pure metabolic (wine red), metabolic (red).

### DETAILED DESCRIPTION

Disclosed herein are methods, systems, and computer program products for computerized classification and prediction of tissue-specific roles of receptors.

Accordingly, in some embodiments, the present disclosure provides for a computational methodology to define receptor roles in tissues. In some embodiments, the present disclosure provides for identifying correlations within tissues among gene expressions coded to receptors. In some embodiments, the present disclosure provides methods of training a machine learning model to identify receptors with specific functions in a target tissue, and the use of that model to predict receptors with that specific function in the target tissue.

The invention is based on the surprising finding of a new methodology to predict tissue-specific metabolic roles of receptors. Linear SVM and k-NN classifiers were used on a feature space of pathway enrichment analysis scores of receptor-co-expressed modules. The method was applied on subcutaneous adipose expression RNA-seq data derived from the GTEx project. As an initial step, semi-supervised learning and a manual literature review were combined to construct a knowledge base of receptors that exhibit metabolic roles in subcutaneous adipose. The performance of the classifiers was evaluated (accuracy >= 0.9) to show that metabolic receptors can be recognized successfully using this new feature space. The k-NN method unexpectedly provides superior performance when compared to the linear SVM method, using the data. Additionally, 21 new metabolic roles for receptors in adipose were predicted when analysing hundreds of unlabelled receptors.

Machine learning approaches were used on gene expression data (the feature space) for classification of functional classes of genes and to infer protein interaction networks. The approach used herein employs further computation on gene expression data to construct a higher-level feature space and classify the metabolic roles of receptors. The enrichment scores that rate the gene’s co-expression network go beyond relating to a single gene, which makes the system more robust to gene-gene network perturbations and noise, and also reduces the number of features, from thousands of features (genes), into several hundreds of features (pathways), which decreases overfitting and improves the classification accuracy.

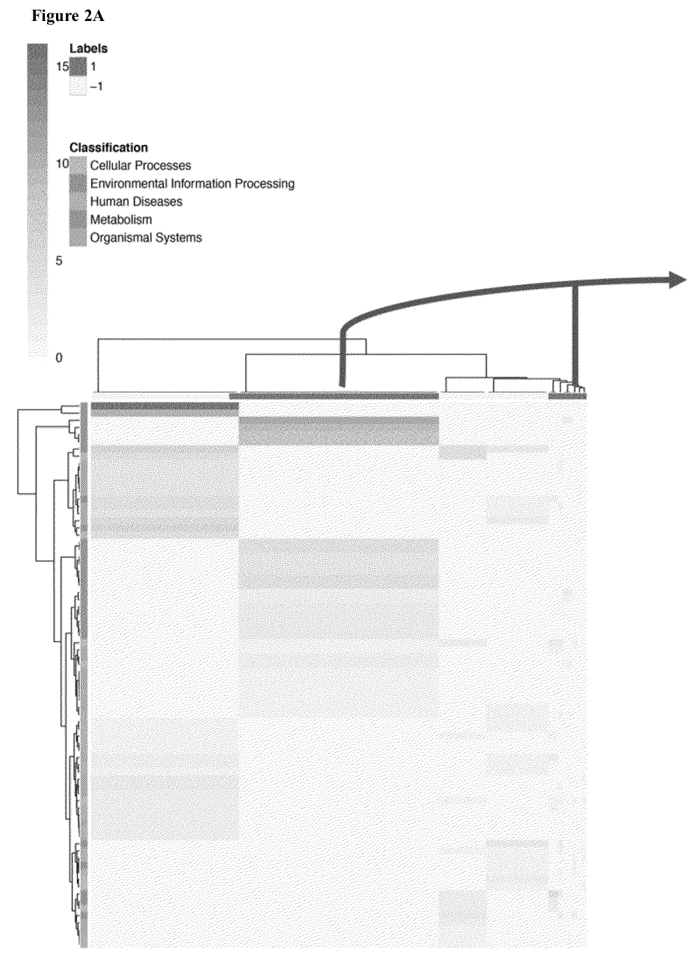

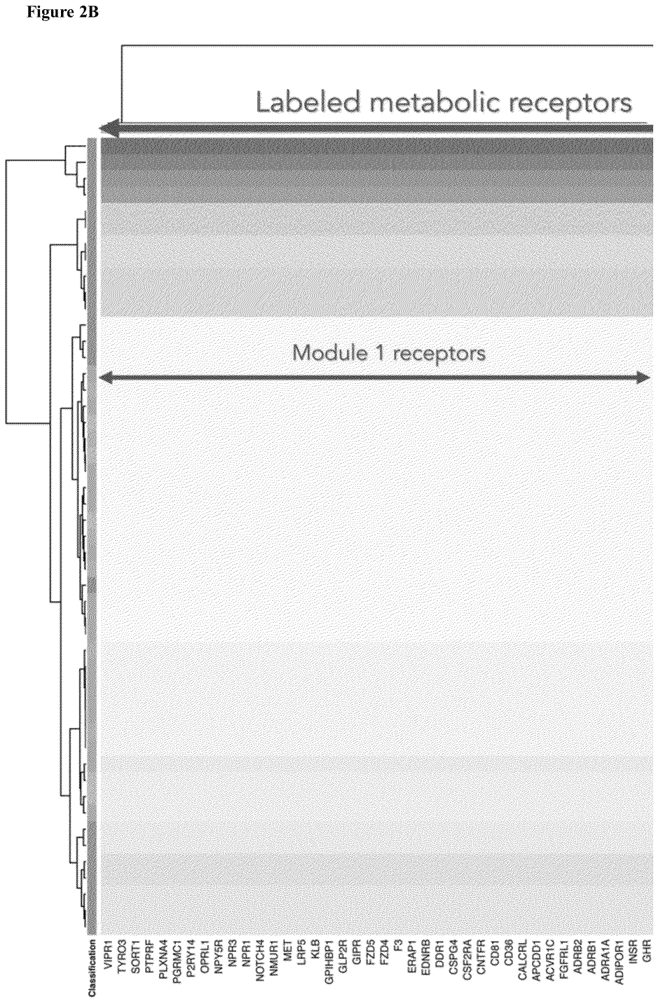

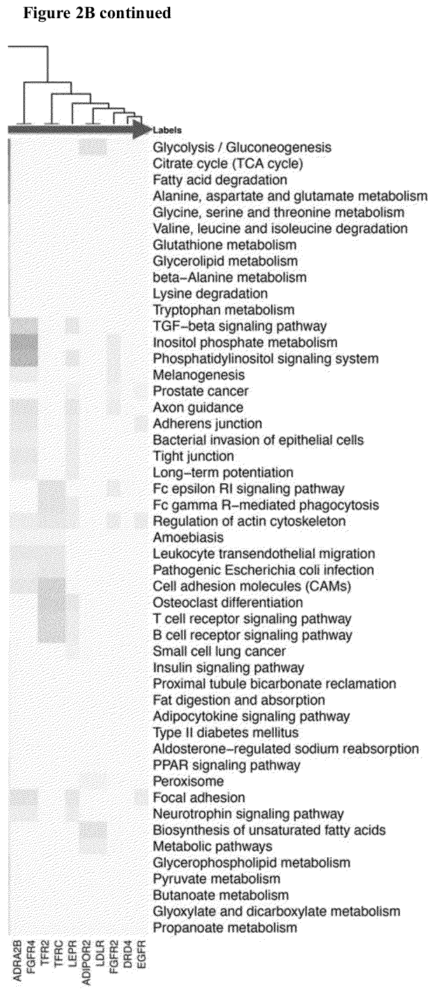

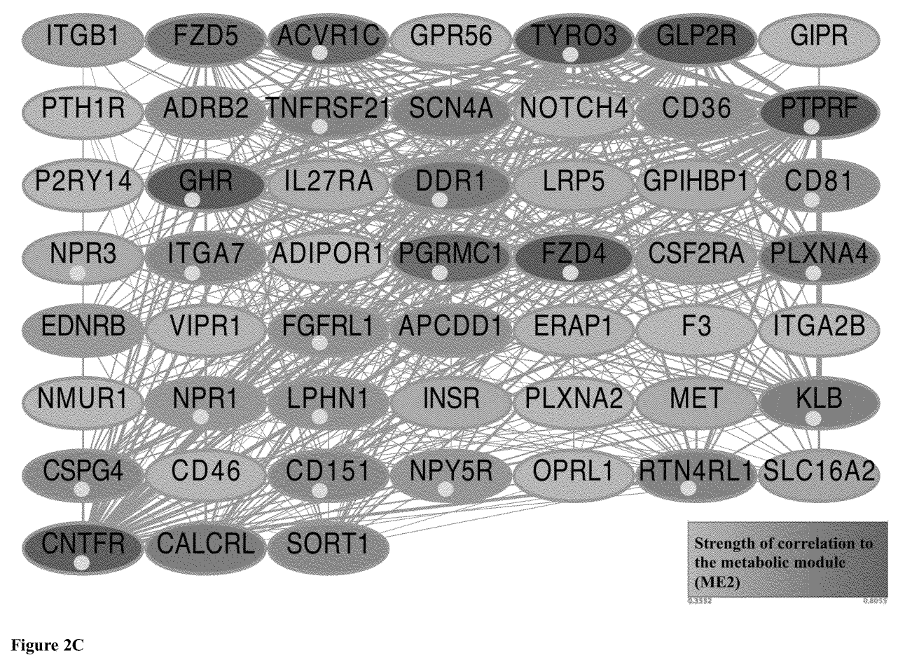

Co-expressed module 1 in subcutaneous adipose is a metabolic module, enriched with multiple metabolic pathways (FIG. 2A) and includes 42 of the labelled metabolic receptors. One can say that metabolic receptors can be detected in an unsupervised manner, just by intuitively extracting the receptors from the metabolically annotated modules, e.g., module 1. So, it is reasonable that the classifiers classify correctly these 42 receptors that are included in module 1. An additional 10 labelled metabolic receptors are included in separate modules (FIG. 2B). Both classifiers classify correctly (rows 1-2 in Table 1) two additional receptors, ADRA2B and FGFR4, which are included in other modules. This approach detects as metabolic these two additional receptors derived from other modules, which is less than intuitive to detect. Moreover, the k-NN classifier detects 8 out of these 10 as being metabolic. The approach is highly accurate in detecting the negative examples as well. When excluding the misclassified negative example, TNFRSF21 cytokine receptor (which may be metabolic in adipose), no false positives (FPs) are detected.

The network-based approach is generalizable and can be used on other tissues. Similar to the approach used for subcutaneous adipose one can perform a thorough literature review directed by a semi-supervised approach, PU SVM bagging, based on multiple classifiers starting from small initial positively annotated examples. The semi-supervised learning defines the negative labels and extends the positive labels, which can be further verified manually using the literature. It was also independently showed that this approach successfully classifies metabolic receptors against a KEGG cytokine receptors list used for negative examples.

In some embodiments, the present disclosure enables detecting biological roles of receptors. In some embodiments, the present disclosure enables detecting tissue-specific biological roles of receptors. In some embodiments, detecting is determining. In some embodiments, the present disclosure enables identifying new correlations between one or more receptors and a biological system, based on coordinated expressions of the new receptors with other proteins that are known members of that system. In some embodiments, the known proteins are known receptors.

In some embodiments, the present disclosure may thus provide for delineating a global mapping of the receptor landscape across the whole human body. In some embodiments, the present disclosure provides a system-level picture of hormonal signaling within the human body. This comprehensive understanding of hormonal signaling pathways would be tremendously significant in many areas of systems biology, drug discovery and modern medicine. Accordingly, the present disclosure may be used to develop new drugs based on newly predicted metabolic receptors and to better understand the side effects of common drugs across different tissue types.

As noted above, the human biological system operates a complex network of communication between organs, to achieve a state of homeostasis. This process is affected through the secretion of ligands (e.g., hormones) into the blood stream from source organs, which then bind to receptors located on both the cell surface and within the cell of target organs. For example, the endocrine systems preserve long feedback loops to maintain glucose or energetic balance, to ensure whole organism survival. Ligands-receptors secretion and binding networks are manifested through small molecules, ligands, secreted into the blood stream from source organs and binding to receptors located on both the cell surface and within the cell of target organs. The networks were found to serve as the information transducers of these loops, which form complex networks of whole-body ligands-receptor interactions. The earlier view of ligands being secreted from one organ and targeting its receptor on another is being replaced with the understanding that this network is far more complex, exhibiting intra-tissue (paracrine) and inter-tissue (autocrine) signaling of ligands targeting distant receptors that are expressed in multiple tissue types.

However, to date, this communication process is only partially understood. Accordingly, in some embodiments, the present disclosure provides for a methodology to predict roles of receptors in tissues with respect to one or more bodily process (e.g., metabolism), based, at least in part, on training a machine learning model using RNA-seq gene expression data.

### Methodology Overview

In some embodiments, the present disclosure provides for a computational methodology to infer tissue-specific roles of receptors across a plurality of human tissue types.

The following discussion will focus extensively, solely as a non-limiting example, on employing the present methodology in a process for identifying the roles of receptors in conjunction with the metabolic system of the human body. However, the present disclosure may be equally effective in predicting and classifying the role of receptors in tissue types in connection with any biological process and/or system, including, but not limited to, the immune system, the endocrine system, the immune system, the circulatory system, the digestive system, the excretory system, the nervous system, the sensory system, the development and regeneration system, the aging, and the like.

In some embodiments, the presently disclosed methodology enables to generate detailed testable hypotheses concerning the metabolic roles of specific receptors in specific tissue types. For example, the present disclosure was applied to identify multiple human metabolic receptors, some of which are known metabolic receptors, e.g., INSR, GHR, while others are novel metabolic receptors with unknown roles, e.g., PLGRKT and LPHN1 (shown to be secreted by human pancreatic islets). When applied, the present disclosure predicted PLGRKT to have multi-tissue metabolic roles in humans, which prediction was validated to be differentially expressed in various metabolic conditions. The present analysis was thus able to identify the metabolic roles of this receptor in almost every human tissue type that was analyzed using the disclosed methodology. In addition, the present methodology also validated PLGRKT to be significantly differentially expressed under metabolic conditions when compared to controls. For example, independent research has found PLGRKT to regulate metabolic homeostasis in a mouse model and promote healthy adipose function. Other research further supports the PLGRKT prediction by showing that PLGRKT exhibits many mutations in metabolic conditions.

In some embodiments, the present disclosure was able to detect house-keeping main metabolic regulators and metabolic receptors which exhibit metabolic roles across many human tissues, based on differential expression in various metabolic conditions. A weak but consistent signal was detected across tissues.

By a first aspect, there is provided a method comprising:
receiving, as input, a dataset comprising gene expression profiles;
applying, to the dataset, gene co-expression network analysis to group the genes into clusters;
applying, to the clusters, a pathways enrichment analysis to assign an enrichment score to the clusters for a pathway of the pathways enrichment analysis, and
identifying a gene encoding a receptor included in a cluster having an enrichment score for a pathway above a predetermined threshold as a receptor that is associated with the biological process of the pathway.

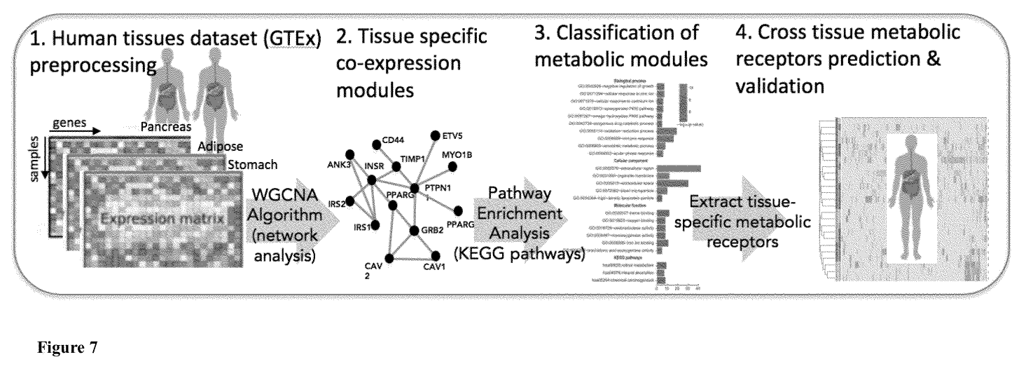

FIG. 7 schematically illustrates an overview of a method for computerized classification and prediction of functions, including tissue-specific functions, of receptors in accordance with some embodiments of the present disclosure.

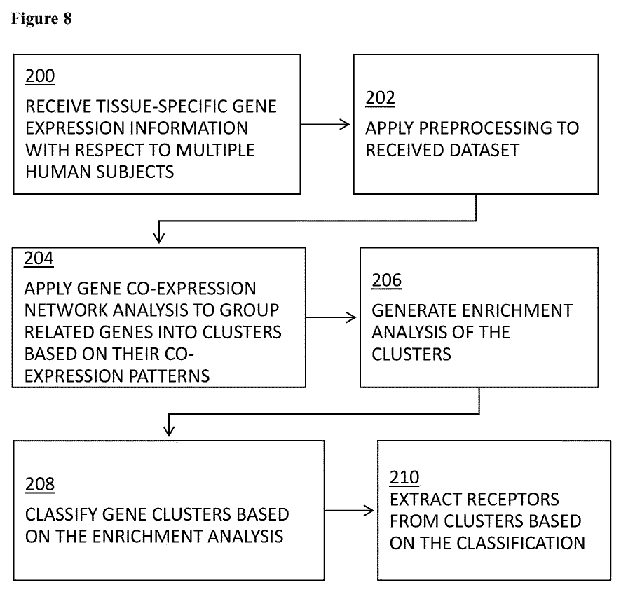

FIG. 8 is a flowchart detailing the functional steps in a process for computerized classification and prediction of tissue-specific or general roles of receptors, in accordance with some embodiments of the present disclosure.

By another aspect, there is provided a method comprising training a machine learning model, on a training set comprising receptors from a first list and receptors from a second list and corresponding labels, wherein the first list of receptors are receptors known to be associated with a biological process and the second list of receptors are receptors known to not be associated with the biological process and wherein the labels comprise an enrichment score for each pathway assigned to a cluster containing a gene encoding that receptor.

By another aspect, there is provided a method comprising:
training a machine learning model, on a training set, the method comprising:
receiving a first list of receptors known to be associated with a biological process and a second list of receptors known to not be associated with the biological process;
receiving a dataset comprising expression profiles in a target tissue for genes encoding proteins, wherein the proteins include receptors of the first list and receptors of the second list;
applying, to the dataset, co-expression network analysis to group the genes into clusters;
applying to the clusters, a pathways enrichment analysis to assign enrichment scores to the clusters for each pathway of the enrichment analysis;
labeling receptors from the first list and receptors from the second list with labels comprising the enrichment score for each pathway assigned to a cluster containing the gene encoding that receptor;
generating a training set comprising receptors from the first list and receptors from the second list and corresponding labels, and
training the machine learning model on the training set to produce a trained machine learning model.

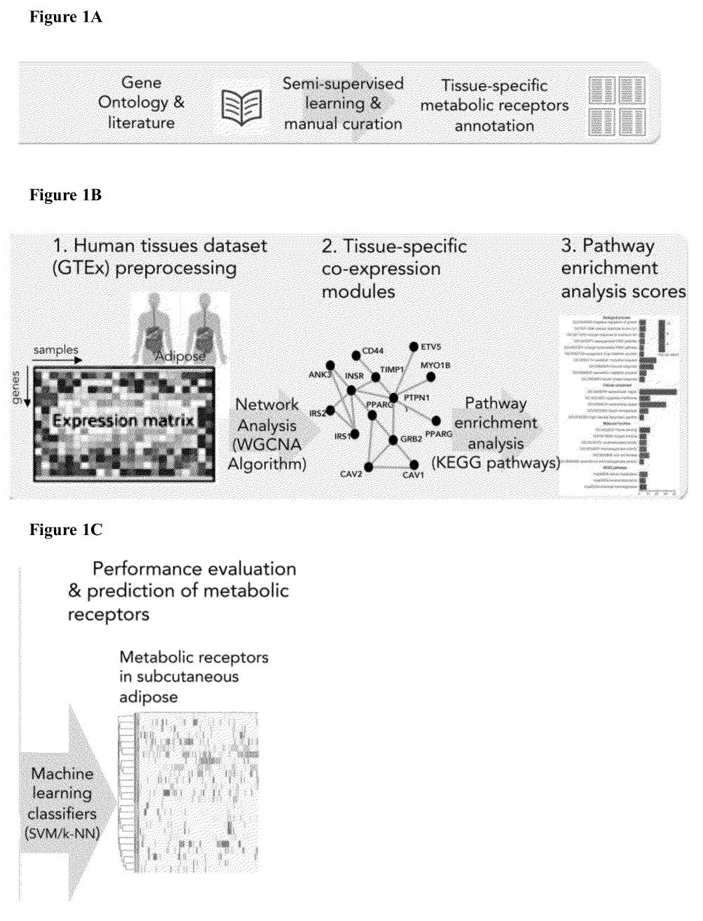

FIG. 1 schematically illustrates an overview of a method for machine learning based classification and prediction of functions, including tissue-specific functions, of receptors in accordance with some embodiments of the present disclosure.

According to another asepct, there is provided a method of determing if a receptor is associated with a biological process, the method comprising:
receiving, as input, a receptor of unkonwn association with the biological process, and enrichment scores for pathways assigned to a cluster of genes containing a gene encoding the receptor of unknown association, wherein the cluster is generated by gene co-expression network analysis; and
applying a trained machine learning model to the input to determine if the receptor of unknown association is associated with the biological process, wherein the trained machine learning model has been trained on a training set comprising a first list of receptors known to be associated with the biological process labeled with labels comprising enrichment scores for pathways assigned to a cluster of genes containing a gene encoding receptors of the first list, and a second list of receptors not associated with the biological process labeled with labels comprising enrichment scores for pathways assigned to a cluster of genes containing a gene encoding receptors of said second list;
thereby determining if a receptor is associated with a biological process in a target tissue.

In some embodiments, at step 200, the present disclosure provides for receiving a dataset comprising gene expressions data. In some embodiments, the gene expression data is gene expression profiles. In some embodiments, the data is tissue-specific data. In some embodiments, the dataset comprises global RNA expression within individual tissue types. In some embodiments, gene expression data is transcriptional data. In some embodiments, gene expression data is mRNA data. In some embodiments, gene expression data comprises a relative expression value for a gene. In some embodiments, gene expression data comprise an absolute expression value for a gene. In some embodiments, the data is for a target tissue.

Numerous methods are known in the art for measuring expression levels of a one or more gene such as by amplification of nucleic acids (e.g., PCR, isothermal methods, rolling circle methods, etc.) or by quantitative in situ hybridization. Design of primers for amplification of specific genes is well known in the art, and such primers can be found or designed on various websites such as http://bioinfo.ut.ee/primer3-0.4.0/ or https://pga.mgh.harvard.edu/primerbank/ for example.

The skilled artisan will understand that these methods may be used alone or combined. Non-limiting exemplary method are described herein.

RT-qPCR: A common technology used for measuring RNA abundance is RT-qPCR where reverse transcription (RT) is followed by real-time quantitative PCR (qPCR). Reverse transcription first generates a DNA template from the RNA. This single-stranded template is called cDNA. The cDNA template is then amplified in the quantitative step, during which the fluorescence emitted by labeled hybridization probes or intercalating dyes changes as the DNA amplification process progresses. Quantitative PCR produces a measurement of an increase or decrease in copies of the original RNA and has been used to attempt to define changes of gene expression in cancer tissue as compared to comparable healthy tissues.

RNA-Seq: RNA-Seq uses recently developed deep-sequencing technologies. In general, a population of RNA (total or fractionated, such as poly(A)+) is converted to a library of cDNA fragments with adaptors attached to one or both ends. Each molecule, with or without amplification, is then sequenced in a high-throughput manner to obtain short sequences from one end (single-end sequencing) or both ends (pair-end sequencing). The reads are typically 30-400 bp, depending on the DNA-sequencing technology used. In principle, any high-throughput sequencing technology can be used for RNA-Seq. Following sequencing, the resulting reads are either aligned to a reference genome or reference transcripts, or assembled de novo without the genomic sequence to produce a genome-scale transcription map that consists of both the transcriptional structure and/or level of expression for each gene. To avoid artifacts and biases generated by reverse transcription direct RNA sequencing can also be applied.

Microarray: Expression levels of a gene may be assessed using the microarray technique. In this method, polynucleotide sequences of interest (including cDNAs and oligonucleotides) are arrayed on a substrate. The arrayed sequences are then contacted under conditions suitable for specific hybridization with detectably labeled cDNA generated from RNA of a test sample. As in the RT-PCR method, the source of RNA typically is total RNA isolated from a tumor sample, and optionally from normal tissue of the same patient as an internal control or cell lines. RNA can be extracted, for example, from frozen or archived paraffin-embedded and fixed (e.g., formalin-fixed) tissue samples. For archived, formalin-fixed tissue cDNA-mediated annealing, selection, extension, and ligation, DASL-Illumina method may be used. For a non-limiting example, PCR amplified cDNAs to be assayed are applied to a substrate in a dense array. Microarray analysis can be performed by commercially available equipment, following manufacturer’s protocols, such as by using the Affymetrix GenChip technology, or Incyte’s microarray technology.

Gene expression data can also be gleaned from a database. Databases with expression for specific organism and/or specific tissues within an organism are well known, and include for example, the Gene Expression Atlas (ebi.ac.uk), the Gene Expression Omnibus (GEO), the Gene Expression Database (GXD, informatics.jax.org), the All of Gene Expression (AOE) database, and the Genotype-Tissue Expression (GTEx) project database to name but a few. In some embodiments, the gene expression data is received from the GTEx database. The GTEx database includes a collection of thousands of samples across multiple tissue types collected from hundreds of donors.

In some embodiments, the tissue is from an organism. In some embodiments, the expression data is from only one organism. In some embodiments, the organism is a mammal. In some embodiments, the mammal is humans. In some embodiments, the expression data is from a plurality of subjects. In some embodiments, the expression data is from at least 1, 2, 5, 10, 20, 30, 40, 50, 100, 200, 300, 400, 500, 600, 700, 800, 900, 1000, 5000 or 10000 subjects. Each possibility represents a separate embodiment of the invention. In some embodiments, the expression data is from at least 100 subjects. In some embodiments, the expression data is from at least 500 subjects. In some embodiments, the expression data is pooled data. In some embodiments, the expression data is the average expression. In some embodiments, the average is the average across subjects.

In some embodiments, the dataset comprises RNA-seq data. In some embodiments, the dataset comprises microarray data. In some embodiments, the dataset comprises PCR data. In some embodiments, the expression comprises the average expression of a gene across cell types within a tissue type, i.e., the sum of all cell type-specific gene expression weighted by cell type proportions within the tissue. In some embodiments, the dataset enables utilizing tissue-level functionality and its networks rather than analyzing a specific cell line at a time, to yield more relevant system-level picture of the functionality of each tissue type and their related receptors co-expression. For example, besides adipocytes, adipose tissue contains endothelial cells, macrophages, and fibroblasts (stromal fraction) that may modulate the overall co-expression patterns of the tissue via crosstalk between the different cell types. Thus, it was shown that factors secreted by the stromal-vascular fraction modulate adipokine secretion by adipocytes, e.g., factors secreted by macrophages have been shown to induce changes in the secretion of adipokines, free fatty acids, and glucose uptake by 3T3-L1 adipocytes. These interactions between cells from the stromal fraction and adipocytes are necessary for physiological functions of adipose tissue diabetes and may affect the expression of genes of each cell type, a signal that may not be detected when analyzing each cell line separately. Therefore, the heterogenous tissue level co-expression networks provide a more relevant information than using solely the cell-specific co-expression network. In some embodiments, the dataset comprises expression data for a whole tissue.

In some embodiments, the dataset comprises expression data for all genes. In some embodiments, all genes are all known genes. In some embodiments, all genes are all annotated gene. In some embodiments, all genes are all genes expressed in a tissue. In some embodiments, all genes are all genes expressed in a target tissue. In some embodiments, all genes are all genes with known functions. In some embodiments, the gene expression profiles are for a plurality of genes. In some embodiments, the gene expression profiles are for at least 100, 200, 300, 400, 500, 600, 700, 800, 900, 1000, 2000, 3000, 4000, 5000, 6000, 7000, 8000, 9000, 10000, 11000, 12000, 13000, 14000, 15000, 16000, 17000, 18000 or 19000 genes. Each possibility represents a separate embodiment of the invention. In some embodiments, the gene expression profiles are for at least 1000 genes. In some embodiments, the gene expression profiles are for at least 15000 genes. In some embodiments, the gene expression profiles are for at least 19000 genes.

In some embodiments, the expression profiles are associated with a corresponding plurality of tissues. In some embodiments, the expression profiles are for expression of the genes in a plurality of tissues. In some embodiments, a plurality of tissues is at least 2, 3, 4, 5, 6, 7, 8, 9, 10, 11, 12, 13, 14, 15, 16, 17 or 18. Each possibility represents a separate embodiment of the invention. In some embodiments, expression profiles are provided for at least 5 tissues. In some embodiments, expression profiles are provided for at least 10 tissues. In some embodiments, the tissues are selected from adipose, muscle, heart, skin, breast, liver, brain, thyroid, esophagus, pancreas, prostate, colon, whole blood, artery, nerve, lung, testis, and pituitary. In some embodiments, adipose is selected from visceral adipose and subcutaneous adipose. In some embodiments, skin is skin that is not exposed to the sun. In some embodiments, esophagus is selected from gastro-esophageal, muscularis and mucosa.

In some embodiments, the dataset comprises, 19,814 protein-coding genes selected from 8555 RNA-seq samples obtained from 544 donors and associated with 53 different human tissue types. In some embodiments, the dataset is the GTEx database (gtexportal.org/home/datasets). In some embodiments, the values in the dataset may be represented as reads per kilobase per million (RPKM) values. In some embodiments, the data is log2 transformed.

In some embodiments, the genes are protein coding genes. In some embodiments, the genes encode proteins. In some embodiments, the proteins are proteins with known functions. In some embodiments, at least one of the proteins is a receptor. In some embodiments, at least one of the genes encodes a receptor. As used herein, the term “receptor” refers to a protein that binds a ligand and transmits a signal upon binding. Ligands can be for example soluble proteins, other receptors, metabolites, minerals and nucleic acids. In some embodiments, the receptor is a cell surface receptor. In some embodiments, the receptor is an internal receptor. In some embodiments, the receptor is a cytoplasmic receptor. In some embodiments, receptors are only cell surface receptors. In some embodiments, the dataset comprises gene expression data for genes that do not only encode receptors. In some embodiments, the dataset comprises gene expression data for genes that encode receptors and non-receptor proteins.

In some embodiments, at step 202, a data preprocessing stage may take place. In some embodiments, data preprocessing may comprise, e.g., outlier removal wherein data outliers may be removed by standardizing samples distances and flagging as outliers the samples with high negative standardized distance (SD < -3) (i.e., more than three standard deviations from the mean). In some embodiments, data preprocessing comprises outlier removal. In some embodiments, the received dataset undergoes a pre-processing step. In some embodiments, the received gene expression data undergoes a pre-processing step. In some embodiments, the pre-processing step corrects for batch effects. In some embodiments, the pre-processing step is as described hereinbelow in the Methods section.

In some embodiments, data from cell types in the same tissue are aggregated into a single tissue category. In some embodiments, the single tissue is the tissue that comprises the cell types. In some embodiments, aggregation of data from the various cell types is relative to the abundance of each cell type in the tissue. In some embodiments, all data associated with various brain tissue types (excluding cerebellum and cerebellar hemisphere) may be aggregated into a single tissue category. In some embodiments, all data associated with blood cell types are aggregated into a single tissue category.

In some embodiments, the data may be subject to confounding factors adjustment. In some embodiments, the confounding factor is cell death. In some embodiments, the confounding factor is subject death. In some embodiments, the type of death classification of the samples (DTHHRDY=death circumstances) is based on 4-point Hardy Scale. Because ventilator death group have significantly shorter ischemic times, which preserve the quality of the samples, the ventilator death group samples were analyzed. It was shown by a previous work by the present inventors that using some of the common methods for adjusting the heterogenous GTEx expression data for hidden confounding factors (e.g., using principal components analysis) filters out much of the biological signals which are relevant for the present analysis. Thus, in the present disclosure, ComBat analysis from the R/Bioconductor package SVA was used to adjust for known confounding factors, that showed superiority. ComBat was applied to adjust for experimental batch, ischemic time (time that elapsed between actual death and sample extraction), gender and age. Due to the discrete nature of ComBat, the continuous ischemic time values were discretized into five bins, labels 1 to 5, by partitioning them into 300 min intervals. Age includes the 20-80 years range and is partitioned into 10 years intervals (embedded in the GTEx dataset). Genes with zero variance per each batch group and type were removed. In addition, batches with one sample within a batch were also removed. Because ComBat is not designed to correct for multiple batch effects simultaneously, each batch was adjusted iteratively, accounting for the yet unadjusted batches in each iteration. Twenty-five human tissue gene expression profiles were successfully corrected by ComBat (for other tissue types there was luck of sufficient sample size per corrected batch).

In some embodiments, ligands and receptors may be linked based, e.g., on a ligand-receptor pair list comprising, e.g., 692 known ligand-receptor pairs (i.e., receptors and their corresponding ligands). In addition, putative ligand-receptor pairs were inferred from PPI databases and text-mining methods.

In some embodiments, at step 204, a co-expression step may be conducted. In some embodiments, one or more suitable correlation networks may be used, e.g., weighted gene co-expression network analysis (WGCNA) for detecting correlation patterns among genes across the tissue-specific samples. In some embodiments, WGCNA may be used for finding clusters (modules) of highly correlated genes, for summarizing such clusters using the module eigengene or an intramodular hub gene, for relating clusters to one another and to external sample traits (using eigengene network methodology), and/or for calculating cluster membership measures. Correlation networks may also facilitate network-based gene screening methods that can be used to identify candidate biomarkers or therapeutic targets.

It will be understood by a skilled artisan that co-expression analysis groups genes based on similar expression profiles or expression levels. Genes that are expressed similarly are grouped or clustered. In some embodiments, the analysis is a machine learning analysis. In some embodiments, the co-expression network analysis comprises employing a WGCNA algorithm. Algorithms, methods and programs for performing co-expression network analysis are well known in the art and any such algorithm, method or program may be used. In some embodiments, the co-expression relationship is determined from the expression profiles. In some embodiments, this step groups related genes into gene clusters based on their co-expression patterns and topological similarity to neighbor genes in the network.

In some embodiments, a suitable tool, such as the WGCNA R software package may be used, wherein the package represents a comprehensive collection of R functions for performing various aspects of weighted correlation network analysis. The package includes functions for network construction, cluster detection, gene selection, calculations of topological properties, data simulation, visualization, and interfacing with external software.

In some embodiments, a suitable algorithm, e.g., a Weighted Gene Co-Expression Network Analysis (WGCNA) algorithm and/or R package may be applied to identify co-expression networks. In some embodiments, an algorithm may be configured to calculate a similarity co-expression matrix using correlation cor(i, j) for all genes. In some embodiments, similarity co-expression calculation may be based, at least in part, on a biweight midcorrelation calculation that accounts for outliers, by assigning larger weights to values closer to medians. In some embodiments, a co-expression matrix of the present disclosure may be transformed into an adjacency matrix by using a soft thresholding power beta (β), to which co-expression similarity is raised:

$${a}_{i j} = \left(0.5 ∗ \left(1 + c o r \left(i , j\right)\right)\right) β$$

where aᵢⱼ represents the resulting adjacency that measures the connection strengths.

In some embodiments, the power β = 12 may be selected based on criteria of approximating scale-free topology of the network. Then, topological overlap matrix (TOM) may be computed and converted into a dissimilarity TOM. TOM may calculate the topological similarity between each two neighbors in the network, i.e., evaluate the similarity of the neighbors for each two nodes. Finally, hierarchical clustering may be used to produce a tree (dendrogram) from the dissimilarity TOM. By using dynamic tree cutting, different number of clusters (modules) may be obtained from the tree. The resulting clusters or clusters may contain genes that are densely interconnected, from which networks may be constructed for each tissue type. “Signed” networks may be used to calculate, with respect to each cluster, the positively or negatively correlated genes, such that the co-expression clusters include positive correlations between the nodes. In some embodiments, a total of 1620 clusters or clusters may be generated across all tissue types. In some embodiments, eigengenes may be defined as the first principal component of the expression matrix for each cluster, which represents the weighted average of the expression profile for each cluster. In some embodiments, the eigengenes may be used to merge clusters and to screen for suitable gene targets by calculating cluster membership (kME) measures, also known as eigengene-based connectivity. This way, key drivers (i.e., genes) may be detected in each cluster.

In some embodiments, the gene co-expression network analysis groups the genes into clusters. In some embodiments, the clusters are tissue-specific clusters. In some embodiments, a given gene is included in only one cluster. In some embodiments, within a given tissue a given gene is included in only one cluster. In some embodiments, a gene appears in a cluster for each tissue in which the gene is expressed. In some embodiments, the clustering is based on a co-expression relationship between the genes. In some embodiments, the clustering is based on a co-expression relationship between the genes within a tissue. In some embodiments, the clustering is based on a co-expression relationship between the genes across tissues. In some embodiments, the tissue is a tissue of the plurality of tissues. In some embodiments, the tissue is a tissue for which expression data was provided.

In some embodiments, at step 206, an enrichment analysis may be performed on the clusters. In some embodiments, the enrichment analysis is a pathway enrichment analysis. In some embodiments, the enrichment analysis is a pathways enrichment analysis.

In some embodiments, the ‘clusterProfiler’ function of the R package may be used to generate enrichment analysis of the clusters. In some embodiments, the R package uses KEGG pathways. In some embodiments, the pathways enrichment analysis comprises KEGG pathways analysis. In some embodiments, KEGG pathways analysis is KEGG pathway analysis. In some embodiments, all 294 KEGG pathways (2016 version) were tested and then filtered for pathways with adjusted p-values < 0.05 (adjusted for multiple corrections using BH (Benjamini & Hochberg) method. In some embodiments, KEGG pathways analysis comprises analysis for all KEGG pathways. In some embodiments, KEGG pathways analysis comprises analysis for a portion of the KEGG pathways. In some embodiments, all pathways are 294 pathways. In some embodiments, at least 10, 20, 25, 30, 40, 50, 60, 70, 75, 80, 90, 95, 97, 99 or 100% of all pathways are analyzed. Each possibility represents a separate embodiment of the invention. In some embodiments, at least 10, 20, 30, 40, 50, 60, 70, 80, 90, 100, 110, 120, 130, 140, 150, 160, 170, 180, 190, 200, 210, 220, 230, 240, 250, 260, 270, 280 or 290 pathways are analyzed. Each possibility represents a separate embodiment of the invention.

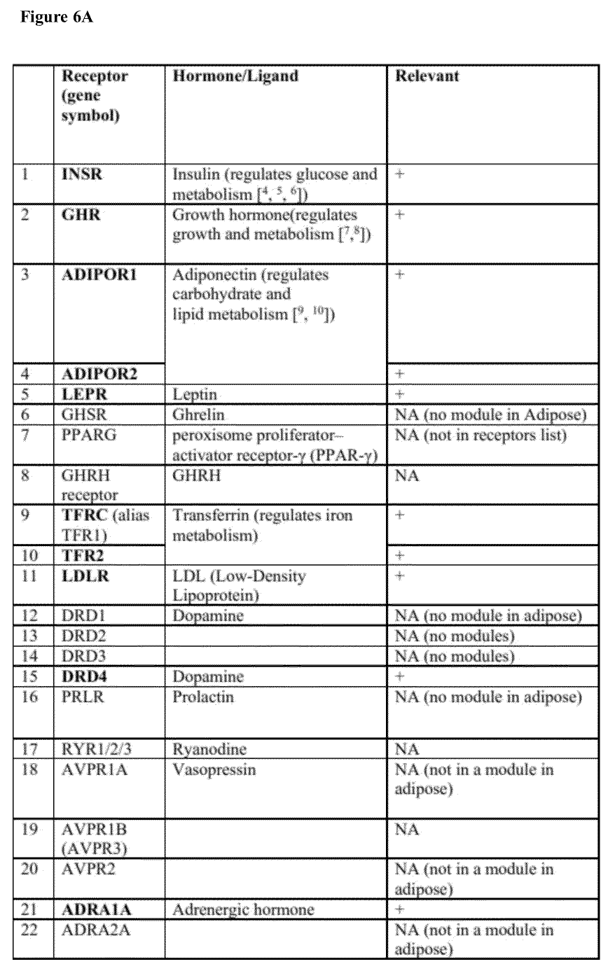

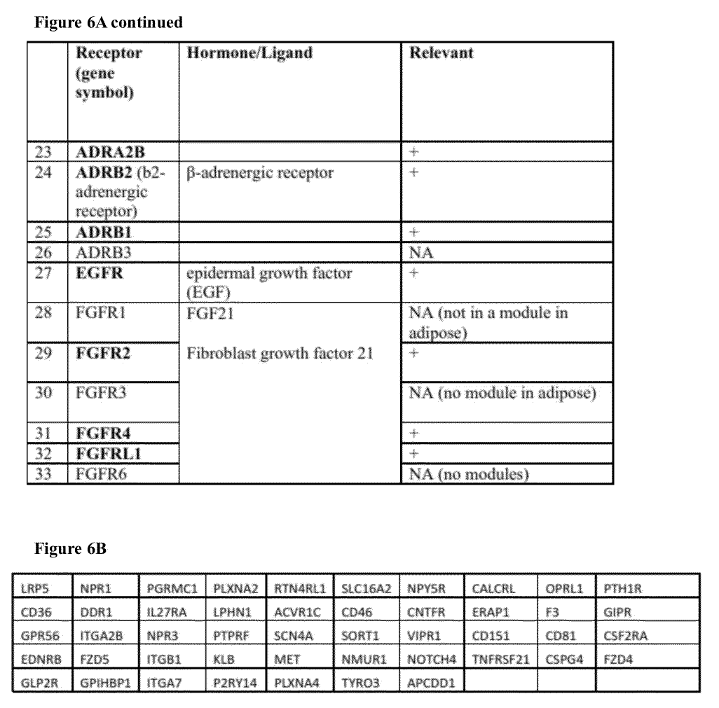

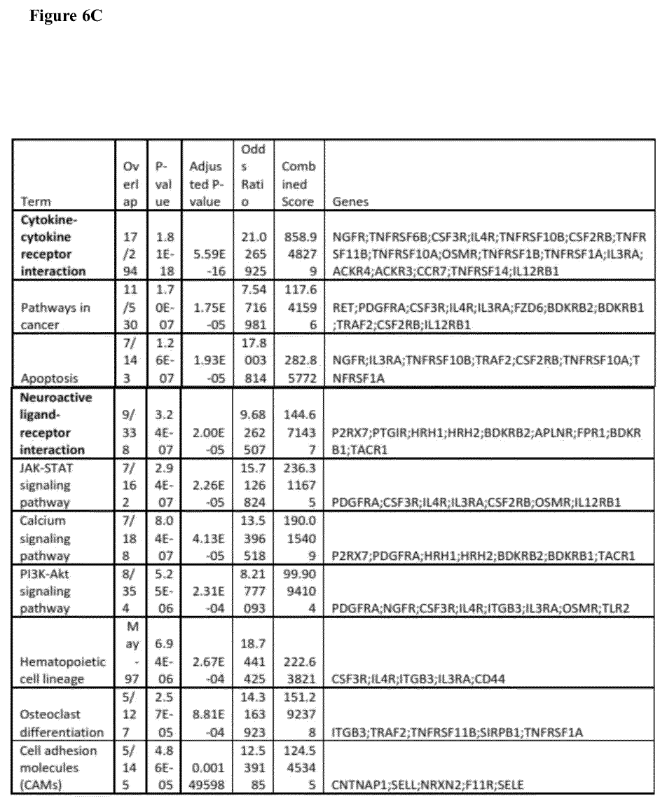

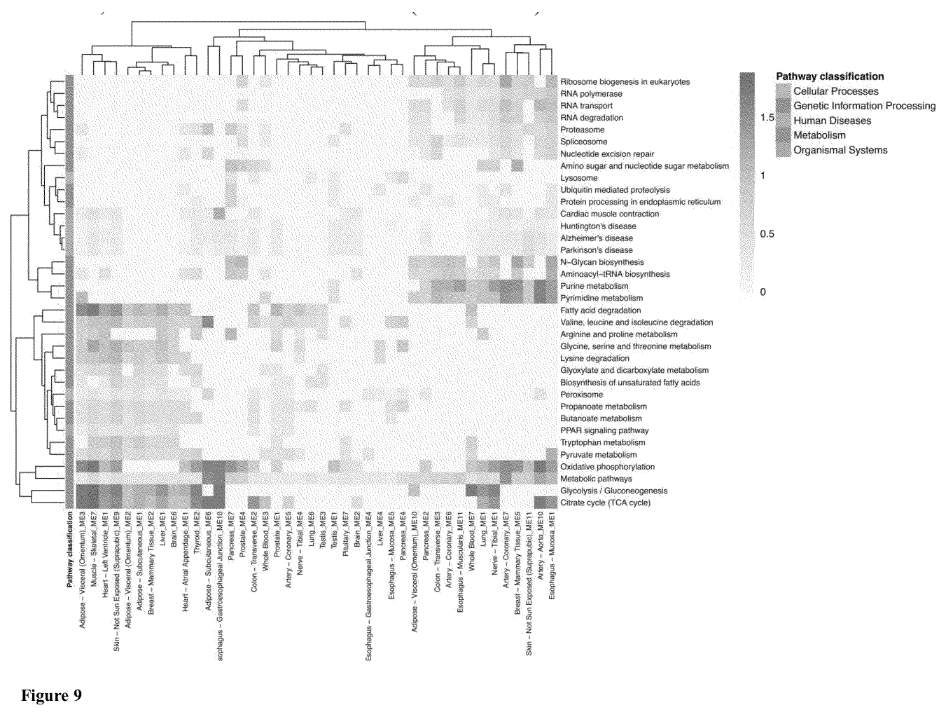

In some embodiments, the pathways are pathways of a biological process. In some embodiments, the pathways are processes. In some embodiments, the processes are biological processes. In some embodiments, the biological process is in a target tissue. In some embodiments, a target tissue is a tissue of interest. In some embodiments, a process is a specific process that is part of a larger class of processes. For example, amino acid metabolism may be said to be part of the larger class of metabolism. Examples of process are provided in FIG. 2B as well as FIG. 6C and FIG. 9. Biological pathways are well known in the art and any pathways of interest may be employed. Examples of pathways include, but are not limited to glycolysis/glucogenesis, TCA cycle, fatty acid degradation, amino acid metabolism, glutathione metabolism, TGF-beta signaling, melanogenesis, prostate cancer, axon guidance, bacterial invasion, adherence junctions, tight junctions, regulation of the actin cytoskeleton, leukocyte migration, cell adhesion, T cell receptor signaling, lung cancer, fat digestion and absorption, type II diabetes, PPAR signaling, the peroxisome, focal adhesion, pyruvate metabolism, and many others.

In some embodiments, 1620 tissue-specific co-expression clusters may be generated by the WGCNA algorithm and analyzed by the KEGG pathways analysis. In some embodiments, a KEGG pathways enrichment analysis may be performed with respect to the clusters. 84 significantly enriched clusters were filtered with the “KEGG_Metabolic_Pathways” pathway. A heatmap of these enrichment analysis results is presented in FIG. 9, where 43 pathways and 43 tissue-specific clusters are presented after further filtering for strongest enrichments and clusters (>5 significant enrichments per column/cluster and >5 clusters per row/pathway). The heatmap values represent the -log transformation of the adjusted p-values enrichment scores.

It can be seen in FIG. 9 that one strong metabolic group may be formed that includes, among others, the pathways “Pyruvate metabolism” and “Glycine, serine and threonine metabolism”, and another metabolic-genetic information processing group. These pathways may be used to classify each cluster as “pure metabolic,” “metabolic,” or “genetic metabolic.”

In some embodiments, pathway enrichment analysis produces an enrichment score. In some embodiments, the enrichment score is an enrichment score for the pathway. In some embodiments, the enrichment score is for the cluster. In some embodiments, each cluster receives an enrichment score for each pathway. In some embodiments, a pathway which is not enriched in the cluster receives an enrichment score of zero. In some embodiments, the enrichment score is for an entire cluster. In some embodiments, the enrichment score is for an entire cluster and not for an individual gene. In some embodiments, the enrichment score is for an individual gene. In some embodiments, the enrichment score is proportional to the magnitude of enrichment. That is the more a cluster is enriched for given pathway the higher the score will be. It will be understood by a skilled artisan that enrichment is an enrichment of genes known to be involved in the pathway. The invention is based at least in part on the surprising finding that genes in a cluster, even though they may not be themselves annotates as part of a pathway are still a part of that pathway based on the fact that they are so clustered. Thus, a cluster may have 20 genes that are related to a given pathway and so the cluster receives a high enrichment score for that pathway. The other genes, and in particular the receptors, in that cluster which may not have contributed to the score are nonetheless indicates to be involved in this same biological pathway. That is, by virtue of the fact that a gene encoding a receptor clusters by expression in a given tissue with other genes involved in certain function then so to this gene encoding a receptor must also be involved in the function.

In some embodiments, at optional step 208, the clusters generated at step 204 and analyzed at step 206, may be classified based on the results of the pathway enrichment analysis. In some embodiments, the classification may be into two or more categories associated with the enrichment values associated with the function of the related biological system. For example, in a metabolic function enrichment model, the clusters may be classified into, e.g., the following categories: “Pure metabolic” clusters which includes metabolic pathways that were clustered together (see FIG. 9) and annotated as related to metabolism by KEGG annotation; “metabolic” clusters; and a “metabolic genetic information processing” group, which comprises genetic information processing by KEGG classes and KEGG BRITE hierarchical classification systems. In some embodiments, a cluster is classified as a metabolic cluster. In some embodiments, the cluster is classified based on a pathway for which the cluster has an enrichment score over a predetermined threshold. In some embodiments, the cluster is classified based on a pathway for which the cluster has an enrichment score that is statistically significant. Thus, for example, if a cluster has a significant enrichment for TGF-beta signaling the cluster is classified as a TGF-beta signaling cluster. A cluster may have more than one classification, just as a protein may have more than one function. Indeed, clusters predominantly have more than one classification.

In other examples, fewer or more categories may be selected. Accordingly, the pathways enrichment results in these metabolic groups were used to classify each cluster as being “pure metabolic,” “metabolic,” or “genetic information processing metabolic.” For each cluster, if the number of pathways in the pure metabolic group was higher than the number of pathways in the second group, it was classified as “pure metabolic” and vice versa. The same may be done for any biological process.

In some embodiments, at step 210, one or more receptors may be analyzed, and their function predicted based on the results of the classification of step 208. As such, the classification given to the cluster may be given to the receptor encoded by a gene found in that cluster. For example, all receptors from one or more categories classified in step 208 may be extracted and identified as associated with a specified biological system of process. For example, all receptors associated with the any category classified as “metabolic” (e.g., “pure metabolic,” “metabolic,” or “genetic information processing metabolic” categories).

FIG. 2C presents receptors derived from a metabolic cluster in Adipose Subcutaneous tissue, and their correlation with the module eigengene. The module eigengene corresponds to the first principal component of a given cluster and considered the most representative gene expression in a cluster. FIG. 2C thus reflects key driver receptors of the metabolic cluster (ME1) in Adipose Subcutaneous. The color tone represents receptor key drivers of the cluster, i.e., the strength of the correlation between the receptor and the eigengene of the metabolic cluster (i.e., the first principal component of the cluster). Edge width represents the weight of receptors co-expressions values based on correlation values and topological similarity (TOM, adjacent nodes that have similar neighbors) between the receptors.

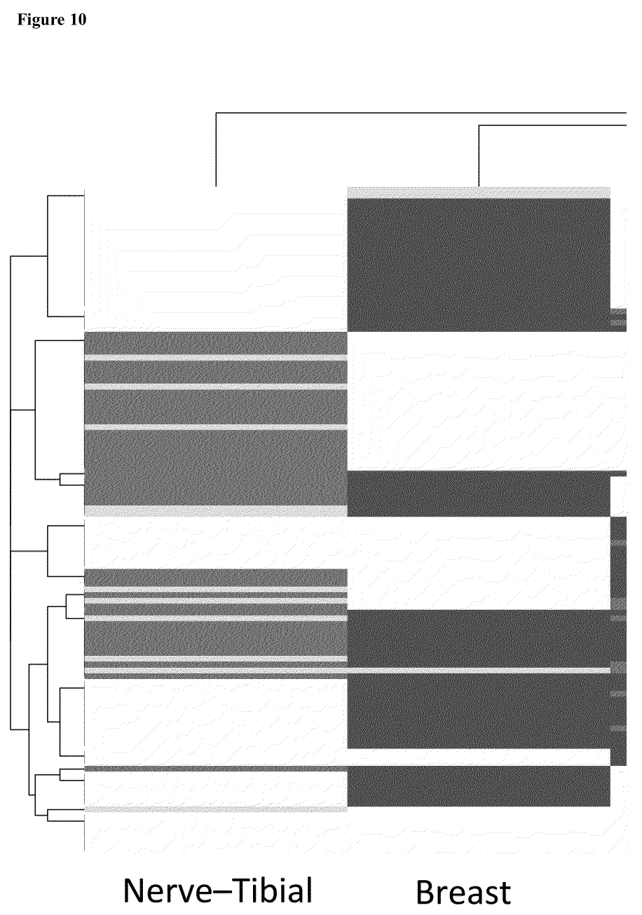

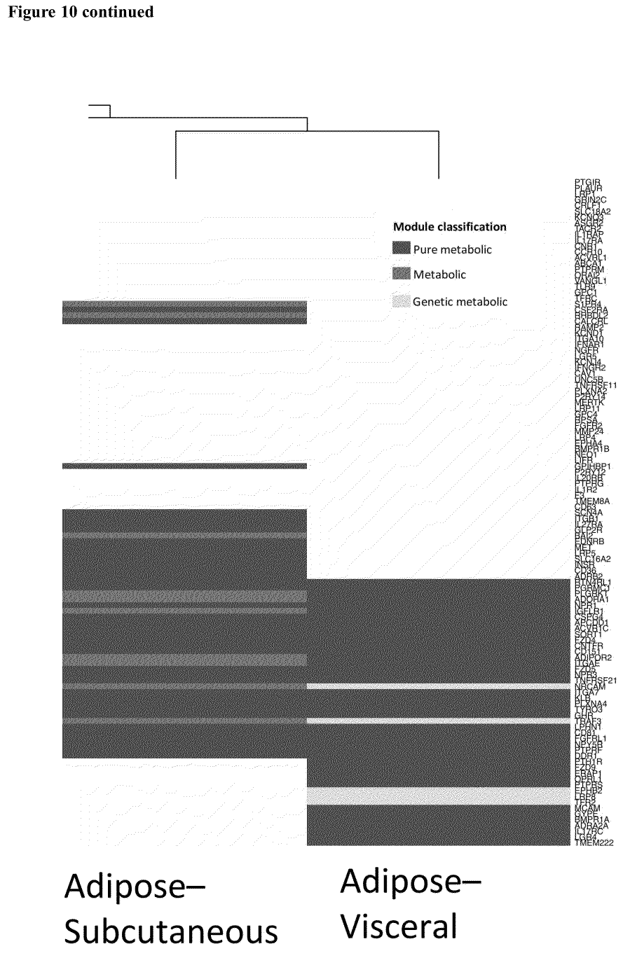

FIG. 10 illustrates the correlation of the receptor with the module eigengene (y-axis), and the x-axis represent the receptors. It can be seen that hormones such as growth hormone (receptor: GHR) and Adiponectin (receptor: ADIPOR2) (0.82 and 0.7 respectively) are highly correlated with the metabolic cluster. Insulin (receptor: INSR) is also correlated strongly to the cluster (correlation is 0.49). it may be noted that the metabolic co-expression cluster and its eigengene may be calculated from all genes (19,814 protein coding genes). C. 527 receptors are metabolic in at least one tissue, across 25 tissues including brain. After filtering for receptors that are metabolic below 3 tissues (leaving receptors that are metabolic in at least 4 tissues) 181 receptors were obtained.

In some embodiments, at least one gene is identified. In some embodiments, the identified gene is a gene encoding a receptor. In some embodiments, receptor encoded by the gene is identified. In some embodiments, the gene is identified as encoding a receptor that is associated with a biological pathway. In some embodiments, the receptor is identified as being associated with a biological pathway. In some embodiments, the gene is identified as encoding a receptor that is associated with a process. In some embodiments, the receptor is identified as being associated with a process. In some embodiments, the process is a biological process. In some embodiments, the process is a process of the pathway. In some embodiments, a plurality of genes is identified. In some embodiments, a plurality of genes is at least 2, 3, 4, 5, 6, 7, 8, 9, 10, 15, 20, 25, 30, 35, 40, 45 or 50 genes. Each possibility represents a separate embodiment of the invention.

In some embodiments, the at least one gene is a gene that encodes a receptor. In some embodiments, the identified gene is a gene within a cluster. In some embodiments, the identified gene is within at least one cluster. In some embodiments, the cluster is a cluster having an enrichment score for a pathway. In some embodiments, the enrichment score is a statistically significant enrichment score. In some embodiments, the enrichment score is an enrichment score above a predetermined threshold. In some embodiments, the enrichment score is an enrichment score exceeding a predetermined threshold. In some embodiments, a score above a predetermined threshold is a statistically significant score. In some embodiments, a gene in a cluster which has a score above a predetermined threshold for a given pathway is identified as encoding a receptor associated with that pathway. In some embodiments, a receptor associated with a pathway is a receptor of the pathway. In some embodiments, a receptor associated with a pathway is a receptor that functions in the pathway.

In some embodiments, a gene is identified that is in more than one cluster with an enrichment score for a particular pathway. In some embodiments, the enrichment score is a score above a predetermined threshold. It will be understood by a skilled artisan that each gene is only in one cluster for a given tissue, but since expression data from a plurality of tissues are examined, a gene will be in multiple clusters (one for each tissue). Thus, if a gene appears in multiple clusters (i.e., in multiple tissues) that all are associated (have an enrichment score above a predetermined threshold) with a particular pathway then that gene is identified as being associated with that pathway. In some embodiments, an identified gene is in at least 2, 3, 4, 5, 6, 7, 8, 9 or 10 clusters with an enrichment score for a particular pathway. Each possibility represents a separate embodiment of the invention. In some embodiments, an identified gene is in at least 3 clusters with an enrichment score for a particular pathway. In some embodiments, an identified gene is in at least 5 clusters with an enrichment score for a particular pathway. In some embodiments, an identified gene is in at least 10 clusters with an enrichment score for a particular pathway. In some embodiments, a particular pathway is a target pathway. In some embodiments, a particular pathway is a pathway of interest. In some embodiments, the identifying comprises identifying genes encoding receptors included in at least 1, 2, 3, 4, 5, 6, 7, 8, 9, or 10 clusters having an enrichment score for a pathway of the specific biological process above a predetermined threshold. Each possibility represents a separate embodiment of the invention. In some embodiments, the identifying comprises identifying genes encoding receptors included in at least 2 clusters having an enrichment score for a pathway of the specific biological process above a predetermined threshold. In some embodiments, the identifying comprises identifying genes encoding receptors included in at least 3 clusters having an enrichment score for a pathway of the specific biological process above a predetermined threshold. In some embodiments, the identifying comprises identifying genes encoding receptors included in at least 5 clusters having an enrichment score for a pathway of the specific biological process above a predetermined threshold. In some embodiments, the identifying comprises identifying genes encoding receptors included in at least 10 clusters having an enrichment score for a pathway of the specific biological process above a predetermined threshold.

In some embodiments, the identified gene is a gene that was not known to be associated with the biological pathway. In some embodiments, the identified gene is a gene that was not known to be associated with the biological process. In some embodiments, the identified gene has a correlation to an eigengene of the cluster above a predetermined threshold. In some embodiments, the identified gene has a correlation to an eigengene of the cluster below a predetermined threshold. In some embodiments, the identified gene is not a gene that contributed to the cluster enrichment score. In some embodiments, the identified gene is a gene that contributed to the cluster enrichment score. In some embodiments, the identified gene is not a gene that significantly contributed to the cluster enrichment score. In some embodiments, the identified gene is a gene that significantly contributed to the cluster enrichment score. In some embodiments, the identify comprises identifying genes encoding receptors with correlation to an eigengene of the cluster above a predetermined threshold. In some embodiments, the identify comprises identifying genes encoding receptors with correlation to an eigengene of the cluster below a predetermined threshold.

In some embodiments, the identifying comprises applying a machine learning model. In some embodiments, the machine learning model is a machine learning model of the invention. In some embodiments, the machine learning model is a machine learning model trained by a method of the invention. In some embodiments, the machine learning model is a machine learning model is trained on receptors associated with the specific biological process and receptors not associated with the specific biological process. In some embodiments, the machine learning model is a machine learning model is trained on enrichment scores. In some embodiments, the enrichment score are enrichment scores of the genes encoding the receptors associated with the specific biological process and receptors not associated with the specific biological process. In some embodiments, the enrichment score are enrichment scores of the clusters containing the genes encoding the receptors associated with the specific biological process and receptors not associated with the specific biological process.

In some embodiments, the machine learning model is trained to predict receptors associated with a biological process. In some embodiments, the biological process is in a target tissue. In some embodiments, the method is a method of predicting receptors associated with a biological process. In some embodiments, the method is a method of determining if a receptor is associated with a biological process. In some embodiments, a biological process is a biological pathway.

Machine learning models are well known in the art and any such model may be used. Models include, but are not limited to artificial neural networks, support vector machines (SVM) classifier and a k-nearest neighbor (k-NN) classifier. In some embodiments, the machine learning model is an SVM classifier. In some embodiments, the machine learning model is a k-NN classifier. In some embodiments, the machine learning model is selected from an SVM classifier and a k-NN classifier.

In some embodiments, the first list of receptors are receptors known to be expressed in the target tissue. In some embodiments, the second list of receptors are receptors known to be expressed in the target tissue. In some embodiments, the receptors of the first list have been experimentally confirmed to be associated with the biological process. In some embodiments, the receptors of the first list are known in the literature to be associated with the biological process. In some embodiments, the receptors of the second list are known in the literature to not be associated with the biological process. In some embodiments, the receptors of the second list have been experimentally confirmed to not be associated with the biological process. In some embodiments, the method comprises performing the experiment to confirm the association or lack thereof. In some embodiments, receptors of the second list are determined using a machine learning algorithm. In some embodiments, receptors of the second list are determined using a Positive unlabeled (PU) support vector machines (SVM) bagging algorithm. In some embodiments, the machine learning algorithm is a PU SVM bagging algorithm.

In some embodiments, the first list comprises at least 2, 5, 10, 15, 20, 25, 30, 35, 40, 45, 50, 60, 70, 80, 90 or 100 receptors. Each possibility represents a separate embodiment of the invention. In some embodiments, the second list comprise at least 2, 5, 10, 15, 20, 25, 30, 35, 40, 45, 50, 60, 70, 80, 90 or 100 receptors. Each possibility represents a separate embodiment of the invention.

In some embodiments, the dataset comprises expression profiles for genes that encode proteins that are not receptors. It was unexpected found by the inventors that when only expression profiles for receptors were used the clusters were insufficiently robust and did not provide statistically significant enrichment data. As such it was determined that in order to receive informative results there was a need to include genes that do not encode receptors. In some embodiments, the dataset comprises less than 50, 40, 30, 25, 20, 15, 14, 13, 12, 11, 10, 9, 8, 7, 6, 5, 4, 3, 2 or 1% genes that encode receptors. Each possibility represents a separate embodiment of the invention. In some embodiments, the dataset comprises less than 50% genes that encode receptors.

In some embodiments, the dataset comprises expression profiles for genes encoding all the proteins of the first list. In some embodiments, the dataset comprises expression profiles for genes encoding all the proteins of the second list. In some embodiments, the dataset comprises expression profiles for all genes expressed in a target tissue. In some embodiments, the dataset comprises expression profiles for all genes expressed in a specific tissue.

In some embodiments, the grouping is based on a co-expression relationship between the genes. In some embodiments, the clusters are based on a co-expression relationship between the genes. In some embodiments, the co-expression between the gene is in the target tissue. In some embodiments, genes in a cluster share a common expression profile. In some embodiments, genes in a cluster are co-expressed. In some embodiments, common expression is similar expression. In some embodiments, common expression is the same expression. In some embodiments, the commonality of expression is determined by the co-expression network analysis. In some embodiments, gene expression analysis is of expression profiles of a set of genes. In some embodiments, the set of gene are expressed in the target tissue. In some embodiments, the set of genes includes genes encoding receptors of the first list. In some embodiments, the set of genes includes genes encoding receptors of the second list. In some embodiments, the set of genes includes a gene encoding the receptor of unknown association.

In some embodiments, each pathway is a pathway of a specific biological process. In some embodiments, at least one of the pathways is a target pathway. In some embodiments at least one of the pathways is a pathway of a target process. In some embodiments, the label comprises an enrichment score for each pathway. In some embodiments, the label comprises an enrichment score for each pathway for which the score is not zero. In some embodiments, the label is for a specific gene but comprises the enrichment scores of the cluster. In some embodiments, all genes in a given cluster have the same label. In some embodiments, the label further comprises gene specific enrichment scores. In some embodiments, a gene specific enrichment score comprises the magnitude of a given genes contribution to the eigengene of the cluster. In some embodiments, a gene specific enrichment score comprises the magnitude of a given genes contribution to the eigengene of the pathway. It will be understood that one specific label will correspond to each receptor, and that every receptor will have its own label. In some embodiments, the label is receptor specific. The label will bear the scores for a specific receptor, which is the scores of the cluster containing the gene that encodes that specific receptor.

In some embodiments, the training set is an annotated training set. In some embodiments, the training set comprises the receptor names. In some embodiments, a receptor is the receptors name. In some embodiments, the gene and the receptor have the same name. In some embodiments, the receptor and the gene encoding the receptor are both identifiable by the receptors name. In some embodiments, the training set comprises all the receptors from the first list. In some embodiments, the training set comprises all the receptors from the second list. In some embodiments, the training set comprises the labels that correspond to each receptor. In some embodiments, the training set comprises the labels that correspond to each receptor of the first list. In some embodiments, the training set comprises the labels that correspond to each receptor of the second list. It will be understood by a skilled artisan that the label for a given receptor contains enrichment scores for a cluster, that is the cluster that contains the gene that encodes that given receptor.

In some embodiments, the method further comprises at an inference stage, receiving, as input, a receptor absent from the training set. In some embodiments, a receptor absent from the training set is a receptor of unkonwn association with the biological process. In some embodiments, a receptor absent from the training set is a receptor of unkonwn association with the biological process in a target tissue. In some embodiments, a receptor absent from the training set is a receptor absent from the first list. In some embodiments, a receptor absent from the training set is a receptor absent from the second list. In some embodiments, a receptor absent from the training set is a receptor encoded by a gene that is not used as part of the pathways enrichment analysis to define a given pathway. In some embodiments, a given pathway is a target pathway. It will be understood by the skilled artisan that the receptor being investigated or tested at the inference stage will not be a receptor that is assigned as part of the pathway of interest by the pathways analysis program/software.

In some embodiments, the inference stage input further comprises enrichment scores for pathways assigned to a cluster of genes containing a gene encoding the receptor absent from the training set. In some embodiments, the cluster of genes containing the gene encoding the receptor absent from the annotated training set is generated by co-expression network analysis. In some embodiments, co-expression network analysis is performed on expression profiles of genes. In some embodiments, the expression profiles comprise expression in a target tissue.

In some embodiments, the inference stage further comprises applying the trained machine learning model to the input. In some embodiments, applying the trained model is to identify a receptor associated with the biological process. In some embodiments, applying the trained model is to identify a receptor associated with the biological process in the target tissue. In some embodiments, applying the trained model is to determine if the receptor absent from the training set is a receptor associated with the biological process.

In some embodiments, the enrichment scores for pathways assigned to a cluster of genes containing the gene that encodes the receptor absent from said annotated training set are generated by a method provided herein. In some embodiments, the method is a method of the invention. In some embodiments, the method comprises receiving a dataset comprising expression profiles in the target tissue for genes encoding proteins, wherein the proteins include the receptor absent from the training set, applying, to the dataset, co-expression network analysis to group the genes into clusters, based on a co-expression relationship between the genes in the target tissue; and applying to the clusters, a pathways enrichment analysis to assign enrichment scores to the clusters for each pathway of the enrichment analysis, wherein each pathway is a pathway of a specific biological process.

The present invention may be a system, a method, and/or a computer program product. The computer program product may include a computer readable storage medium (or media) having computer readable program instructions thereon for causing a processor to carry out aspects of the present invention.

By another aspect, there is provided a system comprising:
at least one hardware processor; and
a non-transitory computer-readable storage medium having stored thereon program code, the program code executable by the at least one hardware processor to perform a method of the invention.

By another aspect, there is provided a computer program product comprising a non-transitory computer-readable storage medium having program code embodied therewith, the program code executable by at least one hardware processor to perform a method of the invention.

In some embodiments, a method of the invention is a computerized method. In some embodiments, a method of the invention is a machine learning method.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user’s computer, partly on the user’s computer, as a stand-alone software package, partly on the user’s computer and partly on a remote computer or entirely on the remote computer or server. In the latter scenario, the remote computer may be connected to the user’s computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The description of a numerical range should be considered to have specifically disclosed all the possible subranges as well as individual numerical values within that range. For example, description of a range from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### EXAMPLES

### Methods

Data pre-processing: GTEx RNA-Seq data of 53 human tissues and 8555 RNA-seq samples from 544 donors was downloaded from the GTEx database (v6), and their reads per kilobase per million (RPKM) values were log2-transformed. 19,814 protein-coding genes were retained. Outlier samples were filtered and all genes within each tissue were quantile normalized (to remove background and sample effects). Outlier removal included standardizing sample distances and flagging as outliers the samples with high negative standardized distance (SD < -3), meaning more than three standard deviations from the mean. Genes with zero variance or missing samples were excluded from the calculation (e.g., for Adipose-Subcutaneous 262 genes were excluded). For a given tissue, genes having at least 0.1 RPKM in 80% or more of the samples were retained. All cell lines were removed from the set of tissues.

Confounding factors adjustment: The type of death classification of the samples (DTHHRDY=death circumstances) is based on a four-point Hardy Scale. The ventilator death group samples were analyzed since it had significantly shorter ischemic times, which preserve sample quality. It has been shown that using some common methods for adjusting the heterogenous GTEx expression data for hidden confounding factors (e.g., using principal components) filters out many of the biological signals—which is relevant here. Thus, ComBat41 from the R/Bioconductor package sva41 was used to adjust for known confounding factors, which has been shown to outperform other methods. ComBat was applied to adjust for experimental batch, ischemic time (time that elapsed between actual death and sample extraction), gender and age. Due to the discrete nature of ComBat, the continuous ischemic time values were discretized into five bins, labelled 1 to 5, by partitioning them into 300 min intervals. Age includes the 20-80 year range and is partitioned into 10 year intervals (embedded in the GTEx dataset). Genes with zero variance per each batch group and type were removed. Batches with one sample within a batch were removed. Each batch was adjusted iteratively, accounting for the yet unadjusted batches in each iteration. ComBat successfully corrected the Adipose Subcutaneous gene expression profiles. The ventilator group samples used were ones which had a relatively short ischemic time and less noise present in the data.

Ligand-receptor pair list: A list of 692 known receptors was imported from an external reference (Ramilowski, J. A. et al., 2015, “A draft network of ligand-receptor-mediated multicellular signaling in human.” Nat. Commun. 6, 1-12, herein incorporated by reference in its entirety) which established a comprehensive collection of ligand-receptor pairs by merging (1) multiple dedicated databases, the Ligand-Receptor Partners (DLRP), IUPHAR and Human Plasma Membrane Receptome (HPMR) databases, another (2) 2,117 experimentally supported interactions in the HPRD and STRING databases, which included 1,288 ligand-receptor pairs absent from (1) that was manually curated from the literature. The final curated set contained 2,422 Ligand-Receptor interactions of 692 distinct receptors.

Co-expression module detection: The Weighted Gene Co-Expression Network Analysis (WGCNA) algorithm and relevant R package were used to identify co-expression networks. The algorithm calculated a similarity co-expression matrix using correlation cor(i,j) for all genes (the biweight midcorrelation measure that accounts for outliers, by assigning larger weights to values closer to medians, was used). The co-expression matrix is transformed into an adjacency matrix by using the soft thresholding power beta β, to which co-expression similarity is raised.

$$\text{aij =} \left(\text{0} \text{.5*} \left(\text{1+cor} \left(\text{i,j}\right)\right)\right) β$$

where aij represents the resulting adjacency that measures the connection strengths.

The power β = 12 was defined based on the criterion of approximating the scale-free topology of the network. Then, a topological overlap matrix (TOM) was computed and converted into a dissimilarity TOM. The TOM calculated the topological similarity between every two neighbors in the network, i.e., evaluated the similarity of the neighbors for every two nodes. Finally, hierarchical clustering was used to produce a tree (dendrogram) from the dissimilarity TOM. By using dynamic tree cutting, different numbers of clusters (modules) were obtained from the tree. The resulting modules contained genes that are densely interconnected, to construct co-expression networks, names also modules, per each tissue. To define, in each module, the positively or negatively correlated genes the “signed” networks were used— meaning that the co-expressed modules include positive correlations between the nodes. A total of 1620 modules were generated across all tissues. Eigengenes are defined as the first principal component of the expression matrix for each module and represent the weighted average of the expression profile for each module. The eigengenes can be used to merge clusters and to screen for suitable gene targets by calculating module membership (kME) measures, also known as eigengene-based connectivity. This way the key driver genes were detected in each module. The WGCNA algorithm generated 1620 modules across tissues.

KEGG enrichment analysis of modules: The R package ‘clusterProfiler’ generated enrichment analysis of the modules using KEGG pathways. All 294 KEGG pathways were used in the analysis and filtered for significant pathways with adjusted p-values <0.05 (adjusted for multiple corrections using the BH (Benjamini & Hochberg) method).

Support Vector Machines (SVMs): Binary classification is the process of labelling the members of a given data set to be included in one of two groups on the basis of whether they have some set of similar properties or not. Two sets of examples, one set from each group, usually named as positive and negative examples, should be defined to train a binary classifier. SVM is a binary classification approach that was shown to perform well in a verity of settings. A linear SVM constructs a hyperplane that separates the positive examples and the negative examples, based on their properties, of belonging to some class. Linear SVM is suited for a small number of samples to avoid overfitting. The R package e1071 was used for the SVM computation.

Positive unlabeled (PU) SVM bagging: Supervised learning requires the definition of positive and negative examples for training. In most of the domains acquiring negative examples is more costly than the positive ones and sometimes even not possible. An example where one does not know whether an example is positive or negative are called unlabeled examples. For example, if a receptor was shown experimentally to be metabolic, it is labeled/annotated as a positive metabolic, but one is uncertain and does not have the full knowledge to annotate the non-metabolic receptors, which are the unlabeled receptors. A set of methods called Positive Unlabeled (PU) learning algorithms are designed to achieve the task of learning from a limited set of positive examples and a large set of unlabeled examples, i.e., in the absence of negative examples. Most such methods use classical supervised classification methods such as a support vector machine (SVM) classifier. A successful algorithm for this aim is the PU bagging SVM algorithm where a random subset from the unlabeled set is created and defined as the negative examples, and a classifier is trained from each of the subsets and the known positive examples. Finally, these multiple classifiers are merged to generate a negative and positive rate for each example. More specifically, the algorithm (1) creates a training set by combining all positive data points with a random sample from the unlabeled points, with replacement, (2) builds a classifier from this “bootstrap” sample, treating positive and unlabeled data points as positives and negatives, respectively, (3) apply the SVM classifier for prediction to whatever unlabeled data points were not included in the trained random sample - hereafter called OOB (“out of bag”) points - and record their scores, (4) repeat the three steps above many times and finally assign to each point the average of the OOB scores it has received, i.e., the rate of classifying negative/positive from all predictions of the gene. Only little improvement was seen in simulated and real data above 100 iterations of the algorithm. The bagging SVM algorithm outperforms the state-of-the-art methods for PU learning and successfully discriminates between unlabeled positive and negative examples even when the number of known positives is limited. The initial data consists of a limited positive set of known literature-reviewed metabolic receptors in adipose and unlabeled receptors set and the PU SVM bagging algorithm was used to extend the positive examples (which we verified to be positive) and define the negative examples.

k-nearest neighbors algorithm (k-NN): The k-nearest neighbors algorithm (k-NN) is a distance-based learning used for classification, and is appropriate for binary classification of two classes. The algorithm uses as input the k closest labelled examples in the feature space. The most common distance measure is the Euclidean distance. A data point is classified by a plurality vote of its neighbors, with the data point being assigned to the class most common among its k nearest neighbors (k is a positive integer, typically small). The performance of k-NNs is very sensitive to the choice of k and an optimal k can be selected by various heuristic techniques. A common way of choosing the empirically optimal k is by testing the error rate under a set of possible k values.

Cross Validation: Cross-validation is a method to evaluate the performance of a prediction model on data points that are not used to train the model. A popular method of cross-validations is sub-sampling (k-fold cross-validation). In k-fold cross-validation, as the name suggests, the dataset is randomly divided into k number of non-overlapping sets. During each iteration, one set is used as a test dataset and the rest are used for training the model. The test dataset is predicted by the trained model. This iteration is repeated k times, each time with a different train and test groups, and generates k different classification models. The performance statistics are calculated by summing in each distinct test group the true positives, true negatives, false positives and false negatives.

Performance evaluation: Sensitivity, specificity, accuracy and MCC (Matthews Correlation Coefficient) were used to evaluate the performance of the cross-validation analysis. The mathematical equations to calculate these parameters are as follows:

$$\text{Sensitivity = TP/} \left(\text{TP+FP}\right)$$

$$\text{Specificity=TN/} \left(\text{TN+FN}\right)$$

$$\text{Accuracy =} \left(\text{TP+TN}\right) \text{/} \left(\text{TP+FB+TN+FN}\right)$$

$$\begin{matrix}\text{MCC=} \left(\left(\text{TP*TN}\right) \text{-} \left(\text{FP*FN}\right)\right) / √ \\ \left(\left(\text{TP+FP}\right) \left(\text{TP+FN}\right) \left(\text{TN+FP}\right) \left(\text{TN+FN}\right)\right)\end{matrix}$$

where, TP, TN, FP, FN and MCC represents true positive, true negative, false positive, false negative and MCC, respectively. Sensitivity and specificity correspond to the proportion of correct predictions of positive and negative examples. The overall correctly predicted examples were calculated by using accuracy, which was the arithmetic mean of sensitivity and specificity. The MCC is used as a measure of the quality of binary (two-class) classifications and is suitable for imbalanced datasets. The MCC evaluates the balance between specificity and sensitivity. The MCC value is equivalent to the Pearson’s phi correlation coefficient to represent the correlation coefficient between the trained and predicted values of binary classifications and lies between -1 to 1. A highly successful predictor will have MCC value near to 1, while opposite and random predictions have MCC value -1 and 0, respectively.

Experimental Design: Linear SVM and k-NN classifiers were used on the features space to classify receptors to be “metabolic” and “non-metabolic” in subcutaneous adipose. The features space included 295 KEGG pathway enrichment analysis scores per each receptor, i.e., per each module that includes the receptor.

To construct the receptors labelled list a directing semi-supervised approach, PU SVM bagging, was used to enrich the initial positive examples and generate the negative ones. An initial data set of 17 positive examples (FIG. 6A) of known literature-based metabolic receptors in adipose were used to start. The PU SVM bagging method was used with the number of iterations t = 100 and the number of unlabeled samples k in each iteration as the number of the positive labelled examples k = 17. Each receptor (with predicted positive rate > 0.7 gathered from the 100 iterations/classifiers) was manually verified to be involved in metabolic/growth regulation (FIG. 6B). More specifically a receptor was annotated as metabolic if (1) the receptor or its ligand is related to metabolic/growth regulation GO functions or/and process or (2) an independent experimental evidence validates the receptor to regulate insulin/glucose/metabolism/growth. This way, 35 verified metabolic receptors were added to the initial 17 metabolic receptors. To evaluate the performance of our approach we used two groups as the negative examples, (1) cytokine receptors derived from the independent KEGG pathway “cytokine-cytokine receptor interaction”, and (2) negative examples generated by the PU SVM bagging algorithm. The pathway enrichment of the 55 strongest negative receptors (rate > 0.8) was evaluated using the Enrichr web tool of group 2 (FIG. 6C).

For SVM classification, the optimized “cost” measure was used to yield the smallest error rate, using the tune() function. The R package e1071 was used for the SVM computation.

For k-NN classifier the knn() function from the R “class” library was executed using Euclidian distance. The optimal k value (the number of nearest neighbors) was chosen by evaluating the error rate under each k=1,2,..,10 and setting the maximal k with the lowest error rate.

The performance of the classifiers was tested by using a 10-fold cross-validation. The labelled receptor list was randomly divided into 10 groups. Classifiers were trained by using 9/10 of the data and were tested on the remaining ⅒. This procedure was then repeated 9 more times, each time using a different ⅒ of the genes as a test group. The performance of the classifier was measured by examining how well the classifier identified the positive and negative examples in the test sets. Each receptor in the test set can be categorized as true positive that is a metabolic receptor in the tested tissue; true negative as a non-metabolic; A false positive that is classified by the classifier as a metabolic receptor but is a non-metabolic; false negatives receptor is placed as being non-metabolic by the classifier but is a metabolic receptor. The performance measures and the number of receptors in each of these categories was reported for the learning methods tested and for both negative groups.

Metabolic receptors prediction: The classification of metabolic function of unknown receptors was performed by first training the classifiers on the labeled receptors. The unlabeled receptors were then classified 4 times, each using SVM or k-NN with the two possible negative groups described earlier. A total of 321 unlabeled receptors (unlabeled receptors that were included in a module in adipose sub.) were labeled this way. The receptors that were predicted to be metabolic was classified as positive by each of the 4 models.

Visualization tool: Module visualization (see FIG. 2C) was performed using the Cytoscape tool57, which allows visualization and analysis of networks of biological associations and interactions.

Data availability: The GTEx data is available for download from (gtexportal. org/home/datasets).

### Results

Herein a new computational methodology to predict tissue-specific receptor metabolic functionality is presented. The new methodology is applied to subcutaneous adipose. The methodology incorporates three steps A, B and C (FIGS. 1A-C) and is based on the new finding that metabolic receptors are co-expressed, among themselves and with other genes. In Step A an annotated list of metabolic and non-metabolic receptors in adipose was constructed using a semi-supervised approach and literature-based validation. In Step B we used the (WGCNA) algorithm for co-expression network analysis to generate gene modules (clusters) in subcutaneous adipose followed by their pathways enrichment analysis. The enrichment scores were used to train SVMs and k-nearest neighbor (k-NN) classifiers and compared their performance, in Step C. Finally, the classifiers were used to predict new metabolic receptors, having previously unknown metabolic functions, in adipose. An extensive list of ~700 receptors was used for the full analyses and predictions.

### Example 1: Subcutaneous Adipose Receptor Labelled List

Supervised learning requires an initial labelled list of known metabolic (positive examples) and non-metabolic (negative examples) receptors in a tissue for the training, performance evaluation and construction of the classifier.

Adipose tissue was chosen for study since it is a highly active endocrine and metabolically important organ, with the ability to modulate glucose homeostasis, energy expenditure, lipid metabolism, and peripheral inflammation. In addition, the existing knowledge about its metabolic receptors roles is extensive and, experimentally, it was robustly tested in comparison to other tissues.

One main challenge was to detect the receptors that exhibit metabolic roles in adipose and those that do not. It should be noted that the term metabolic receptors is used to include receptors related to the metabolic/endocytosis/growth regulation system. This knowledge is not easily available since public databases, such as KEGG, do not include a metabolic receptor classification in general or a tissue-specific metabolic receptors classification in particular. For example, the KEGG database includes the “Neuroactive ligand-receptor interaction” pathway that consists of a combination of metabolic and non-metabolic receptors. The insulin receptor is included in its own pathway, the KEGG insulin signaling pathway. In addition to the “pure” metabolic receptors a receptor may exhibit ubiquitous roles across the whole body, e.g., a known inflammation-related cytokine receptor which we possibly label as a non-metabolic negative example, may exhibit metabolic roles in adipose. An example is the cytokine receptor TNFRSF21, a tumor necrosis factor receptor superfamily member 21, that is include in the KEGG “Cytokine-cytokine receptor interaction” but is also related to the “regulation of lipid metabolic process” in GO (Gene Ontology).

To construct the initial positively labelled receptors list, a list of 33 metabolic receptors known from the literature to be related to the regulation of growth, endocytosis and metabolism were gathered. This list is provided in FIG. 6A. After filtering for the receptors not available in adipose (e.g., not included in any module in subcutaneous adipose; see the Methods section), a total of 17 receptors were received as the initial positive example set. To enrich the positive and negative examples set, the SVM PU (positive unlabeled) bagging algorithm was used. This algorithm is suitable to use a limited number of known positive examples to successfully discriminate between the positive and negative examples in an unlabeled data set (see Methods section). This way, an additional 37 metabolic receptors were added as positive examples and were verified manually as being metabolic using the GO (Gene Ontology) database and a thorough literature review (see Methods section and FIG. 6B for the list).

As the negative examples two distinct groups were used: (1) cytokine receptors from the KEGG “Cytokine-cytokine receptor interaction” pathway that were not included in the positively labelled examples (total of 61 receptors) and (2) a total of 55 receptors inferred to be strongly negative (negative rate >0.8 out of 100 experiments, see Methods section) by the PU bagging algorithm. Pathway enrichment analysis on the second group’s receptors shows that they were significantly enriched (adjusted p-value < 10-16) with 17 cytokine receptors from the “Cytokine-cytokine receptor interaction” and nine different neuroactive receptors from the KEGG “Neuroactive ligand-receptor interaction” pathway (FIG. 6C).

### Example 2: Co-Expression Modules and Pathway Enrichment Analysis

The GTEx subcutaneous adipose gene expression data was filtered, pre-processed and corrected for batch effects as described in the Methods section.

A total of 17 modules, co-expression networks, were generated by the WGCNA algorithm (see Methods) for subcutaneous adipose tissue. Following modules construction, pathway enrichment analysis was conducted using KEGG pathways. A heatmap of the pathway enrichment scores of the metabolically annotated receptors in subcutaneous adipose (positive examples) and negative examples (group 2), which was used is presented in FIGS. 2A-B. The presented enrichment scores are the -log10 transformation of the adjusted p-value enrichment scores (see Methods). Rows and columns with zero enrichments were removed from the representation. FIG. 2A shows that the positive metabolically annotated receptors (highlighted in the column’s annotation in green) form a strong cluster annotated by KEGG hierarchies to constitute the metabolic process (highlighted on the row’s annotation to the right in turquois) and to include various metabolic pathways. FIG. 2B focuses on the positive metabolic examples to show their enriched metabolic pathways. These include, among others, “Glycolysis”, “Fatty acid degradation”, “Pyruvate metabolism” and “Glycine, serine and threonine metabolism”. Most metabolic receptors are co-expressed and included in Module 1 of subcutaneous adipose (the left cluster in FIG. 2B), e.g., insulin receptor (INSR), adiponectin receptor 1 (ADIPOR1), and growth hormone receptor (GHR). Module 1 receptors network is presented in FIG. 2C to highlight the receptors’ connectivity and correlation with the module eigengene.

### Example 3: Classifier Construction and Validation

Supervised approaches, linear SVM and k-NN, were used to solve the problem of binary classification of receptors to be “metabolic” and “non-metabolic” in subcutaneous adipose tissue (further elaboration is provided in the Methods). 10-fold cross-validation was used to evaluate the performance of the classifiers. Each of the two negative groups (cytokines and the inferred group) was used separately. The performance of the classifiers for the positive labels and each of the negative labels is presented in Table 1. Columns 4-7 are the false positive (FP), false negative (FN), true positive (TP), and true negative (TN). Columns 8-11 are the sensitivity (Sen.), specificity (Spec.) and accuracy (Acc.) measure of overall performance and the MCC (see Methods). The accuracy of all calculations is >0.9. k-NN outperforms SVM with an accuracy of 0.98 and a Matthews Correlation Coefficient (MCC) measure of 0.96 for the inferred negative group. k-NN classified 50 positive examples (TP) correctly as opposed to 44 for the SVM linear classifier. For the cytokine receptors as the negative group, the classifiers produced similar results with 42 TPs. There were few FPs (<=1), which means that this approach will not label a non-metabolic receptor as a metabolic one. The experiment was repeated with SVM and k-NN ten more times with different random splits of the data. The variance introduced by the random splitting of the data was very small (<10-5) relative to the mean.

Table 1

Comparison of Performance Evaluation of Linear SVM and k-NN classifiers (using the Euclidian distance) for Metabolic Receptors Classification in Subcutaneous Adipose Tissue. The Data Includes the 50 Positive Examples and 55 Negatives Examples for the Bagging and 61 for Cytokines.

| | Method | Negative group | TP | TN | FP | FN | Sen. | Spec. | Acc. | MCC | FP recep -tors | FN receptors |
|---|---|---|---|---|---|---|---|---|---|---|---|---|
| 1 | k-NN | inferred | 50 | 55 | 0 | 2 | 0.96 | 0.96 | 0.98 | 0.96 | NA | TFRC TFR2 |
| 2 | SVM | inferred | 44 | 55 | 0 | 8 | 0.85 | 0.87 | 0.93 | 0.86 | NA | ADIPOR2 DRD4 EGFR FGFR2 LDLR LEPR TFR2 TFRC |
| 3 | SVM/ k-NN | cytokine s | 42 | 60 | 1 | 10 | 0.81 | 0.86 | 0.9 | 0.81 | TNF RSF 21 | ADIPOR2 ADRA2B DRD4 EGFR FGFR2 FGFR4 LDLR LEPR TFR2 TFRC |

TNFRSF21 receptor (Tumor necrosis factor receptor superfamily member 21) is a cytokine receptor that is misclassified as a non-metabolic receptor by both k-NN and SVM. TNFRSF21 is related to the “regulation of lipid metabolic process” in GO and predicted by the analysis to exhibit a metabolic role in adipose. When excluding TNFRSF21, no FPs are detected. TFR2 and TFRC are both misclassified as metabolic receptors by both classifiers.

### Example 4: Metabolic Role Predictions for Unlabeled Receptors

In addition to validating the classification accuracy of the classifiers using the labelled receptor list, the classifiers were used to classify unlabelled receptors. Linear SVM and k-NN models were trained on all the labelled data (for the two negative groups separately, resulting in 4 classifiers) and then the four models were used to predict the unlabelled receptors. A total of 285 unlabelled receptors were included in the analysis, i.e., were incorporated in a co-expressed module in subcutaneous adipose (out of 588). Of these, 21 receptors were predicted by all the four classifiers to exhibit metabolic roles in subcutaneous adipose. Table 2 presents the predicted metabolic receptors.

Table 2

Predicted Metabolic Roles of Unlabelled Receptors by Four Classification Models in Subcutaneous Adipose.

| | Predicted metabolic Receptor | | Predicted metabolic Receptor |
|---|---|---|---|
| 1 | CD151 | 12 | P2RY12 |
| 2 | CD46 | 13 | PLGRKT |
| 3 | CD63 | 14 | PLXNA2 |
| 4 | FZD9 | 15 | PTH1R |
| 5 | GPR56 | 16 | RHBDL2 |
| 6 | IL27RA | 17 | RTN4RL1 |
| 7 | ITGA2B | 18 | SCN4A |
| 8 | ITGA7 | 19 | SDC1 |
| 9 | ITGAE | 20 | SLC 16A2/ MCT8 |
| 10 | ITGB 1 | 21 | TACR2 |
| 11 | LPHN1 | | |

### Example 5: Metabolic Receptors Across Tissues

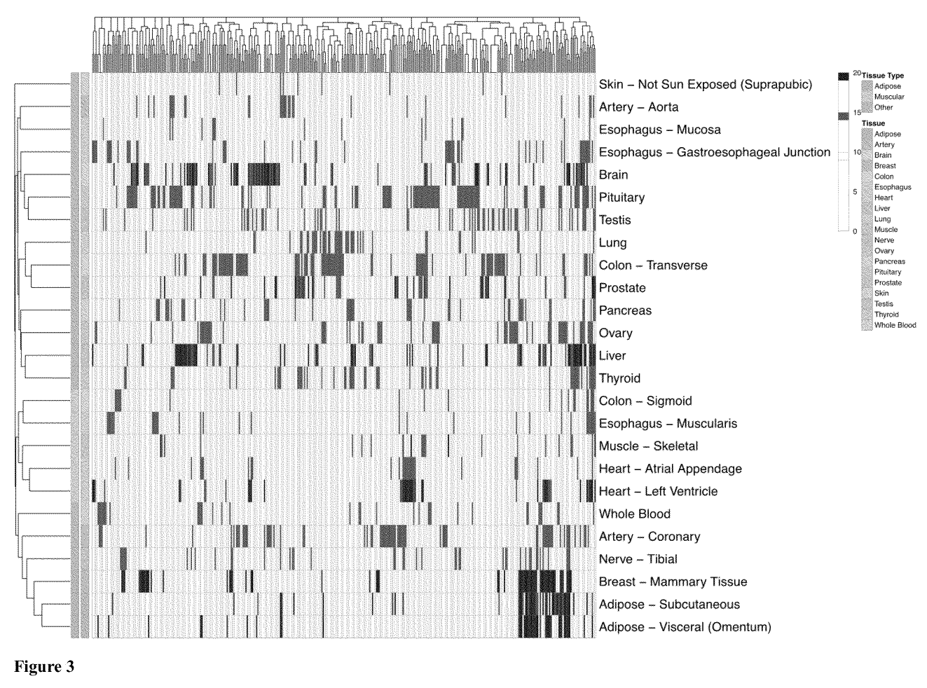

Twenty-five human tissues were clustered by their corresponding metabolic receptors (FIG. 3). Out of 629 receptors identified in the dataset obtained in the present disclosure, 527 receptors are included in at least one metabolic cluster in any tissue type, and 269 receptors may be enriched in 3 or more metabolic clusters (using KEGG “metabolic pathways” enrichment analysis). The present approach predicts that many receptors have metabolic roles in diverse tissues. Of all the receptors, 527 receptors which were identified to have metabolic roles in at least 1 tissue, and 378 receptors have metabolic roles in 2 or more tissues.

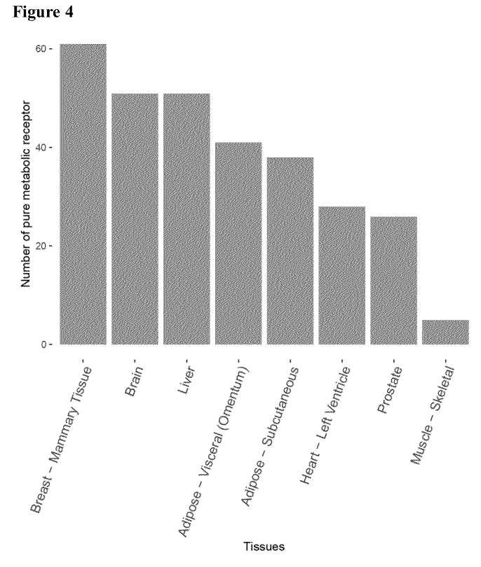

It can be seen in FIG. 3 that tissues are primarily clustered by tissue types, e.g., adipose (“Adipose - Visceral” and “Adipose - Subcutaneous”), heart (“Heart - Atrial Appendage” and “Heart - Left Ventricle”), and secondarily by their cell types. For example, tissues that are composed of adipocyte cells such as, “Adipose Subcutaneous”, “Adipose Visceral” and “Breast - Mammary Gland” have similarity in receptors and are clustered together, while tissues that are composed of muscular cells that include two heart tissues, “Muscle—Skeletal”, “Colon-Sigmoid” and the muscular part of the esophagus, “Esophagus- Muscularis” are clustered together. Tissue receptors that were classified in the “pure metabolic” category are presented in FIG. 4 - breast, brain, liver, adipose, muscle, heart, and prostate, which are all known to be metabolically active tissues. Indeed, subcutaneous adipose tissue is the largest metabolically active organ in the human body, and brain, liver, heart, and muscle are other known metabolically active tissues. The prostate is also related as a metabolically active tissue.

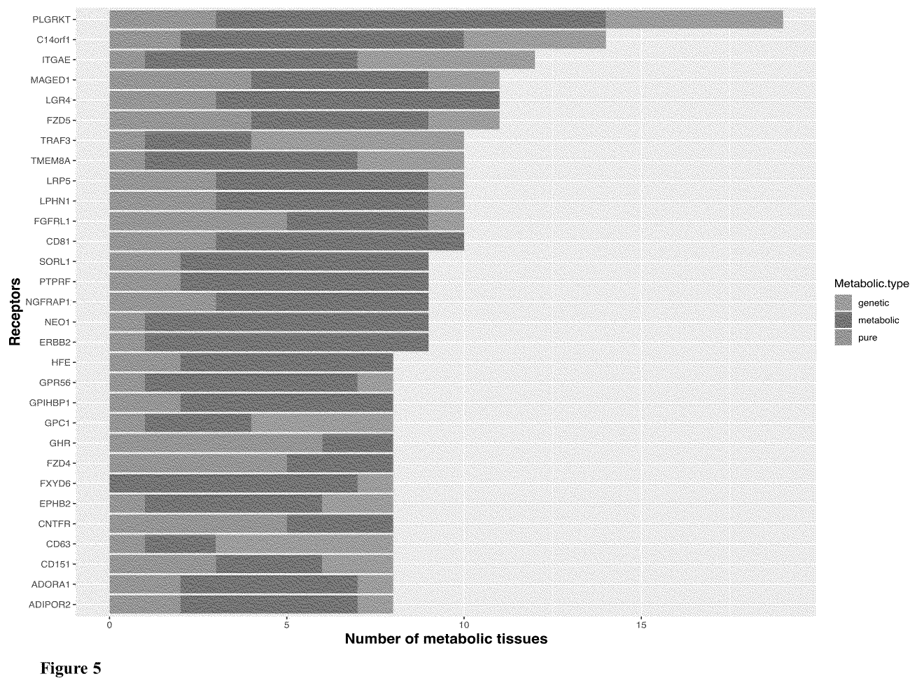

FIG. 5 presents the strongest metabolic receptors that exhibit metabolic roles in most of the examined tissues. Among the strongest cross-tissue metabolic receptors is PLGRKT, a plasminogen receptor. Plasminogen (PLG) is the zymogen (inactive precursor of an enzyme) of plasmin, the major enzyme that degrades fibrin clots. In addition to its binding and activation on fibrin clots, PLG also specifically interacts with cell surfaces where it is more efficiently activated by PLG activators (uPA and tPA) and regulated by Plg activator inhibitor-1 (PAI-⅟SERPINE1). Plasmin exhibits a broad-spectrum proteolysis activity with cell surfaces that functions to promote cell migration during inflammation, wound healing, muscle regeneration and more. PLG has several receptors and the interplay between plasminogen and its receptors known to regulate inflammation. PLGRKT is a newly discovered plasminogen receptor that is highly conserved across mammalian species and broadly expressed in human tissues. It significantly enhances plasminogen activation by supporting binding of plasminogen activators that has a role in macrophage recruitment during inflammatory response. PLGRKT is proposed to be part of a local catecholaminergic cell plasminogen activation system that regulates neuroendocrine prohormone processing. The present results imply that PLGRKT is a main cross-tissue regulator of metabolism. In support of these computational findings, a recent novel study proposed that PLGRKT regulates metabolic homeostasis and promote healthy adipose function in a mouse model. Experimental results showed that Plg-RKT^{-/-} mice gained significantly more weight, and specific Echo MRI measurements showed that total fat mass was significantly greater in Plg-RKT^{-/-} mice compared with Plg-RKT^{+/+} littermates, epididymal adipose tissue weight was significantly less, while liver weights were significantly greater. In insulin signaling studies, Akt phosphorylation was 80% lower in adipose tissue of Plg-RKT⁻¹⁻ mice.

13 of the cross-tissues predicted metabolic receptors were validated to be differentially expressed in metabolically related conditions of humans and mice, e.g., diabetic versus controls and obese versus lean subjects. For this, 16 publicly available gene expression datasets were obtained and filtered from the Gene Expression Omnibus (GEO) repository. Table 3 presents the seven significant datasets (p-values <0.1) received using GSEA algorithm for these 19 receptors. These receptors were differentially expressed in these datasets between control cohorts and metabolically malfunctioned related cohorts.

Table 3

List of 19 Strongest Cross-Tissue Predicted Receptors.

| | GEO dataset and tissue | GSEA p-value | Tissue | Groups (# samples) | Organism (Hs - Homo sapiens/Mm-Mus musculus) |
|---|---|---|---|---|---|
| 1 | GSE29718 | 0.00061 | Adipose visceral | lean (2) obese (3) | Hs |
| 2 | GSE27382 | 0.00069 | Sciatic nerve | diabetic (6) non-diabetic (7) | Mm |
| 3 | GSE15729 | 0.00266 | Aorta | diabetic (8) non-diabetic (7) | Mm |
| 4 | GSE24290 | 0.00538 | Sural nerve | progressive diabetic neuropathy (18) non-progressive diabetic neuropathy (17) | Hs |
| 5 | GSE29718 | 0.04997 | Adipose subcutaneous | lean (8) obese (7) | Hs |
| 6 | GSE25724 | 0.05644 | Pancreatic islets | diabetic (6) non-diabetic (7) | Hs |
| 7 | GSE24031 | 0.06946 | Liver | low fat diet (10) high fat diet (8) | Mm |

The Gene Expression Omnibus (GEO) repository was used to gather high throughput data to perform differential gene expression analysis for a list of “strongest” predicted receptors. To detect relevant gene expression data sets, the keyword “diabetes” was used to search the GEO DataSets homepage (https://www.ncbi.nlm.nih.gov/gds), and the results were filtered by “disease state,” using the “subset” option in GEO search engine. The “disease state” option filtered datasets that compares between diseases conditions of diseased and control groups. Datasets of human and mouse samples were used ( homosapiens and musmusculus respectively), including DataSets starting from 2010 onward. The GEO query “Disease state [Subset Variable Type] diabetes” found a total of 34 DataSets of which 16 were extracted for analysis. Datasets that included unrelated diseases were removed. Then a tool was used to find the homologues of the human genes in mouse (see informatics.jax.org/faq/ORTH_gene.shtml). DEG analysis was performed by dividing the samples into control and cases groups with the GEO2R tool (see ncbi.nlm.nih.gov/geo/info/geo2r.html), which conducts a t-test and uses the limma R package. Then the 10 strongest receptors were selected at each of the following classifications: “pure metabolic,” “pure metabolic + metabolic,” and “all”, which resulted in a list of 19 receptors. The “fgsea” R package and the fgsea() function were then used for conducting GSEA (gene set enrichment analysis) for the list of strongest predicted metabolic receptors. GSEA is a commonly used method for analyzing gene expression data. It allows to select from an a-priori defined list of gene sets those which have non-random behavior in a considered experiment and to identify pathways that contain many co-regulated genes but with small individual effects. Then, the log fold change of each gene was used between the cases and controls and with 10,000 permutations, to calculate the p-values in the GSEA algorithm.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method comprising:
training a machine learning model to predict receptors associated with a biological process in a target tissue, on a training set, the method comprising:
receiving a first list of receptors known to be associated with said biological process and expressed in said target tissue and a second list of receptors known to be expressed in said target tissue and not associated with said biological process;
receiving a dataset comprising expression profiles in said target tissue for genes encoding proteins, wherein said proteins include receptors of said first list and receptors of said second list;
applying, to said dataset, gene co-expression network analysis to group said genes into clusters, based on a co-expression relationship between said genes in said target tissue;
applying to said clusters, a pathways enrichment analysis to assign enrichment scores to said clusters for each pathway of said enrichment analysis, wherein said each pathway is a pathway of a specific biological process;
labeling receptors from said first list and receptors from said second list with labels comprising said enrichment score for said each pathway assigned to a cluster containing said gene encoding said receptor;
generating an annotated training set comprising receptors from said first list and receptors from said second list and corresponding labels; and training said machine learning model on said annotated training set to produce a trained machine learning model.

2. The method of claim 1, wherein said receptors are cell surface receptors, internal receptors within a cell or a combination thereof, said expression profiles are mRNA expression profiles, or both.

3. (canceled)

4. The method of claim 1, wherein said gene co-expression network analysis comprises employing a Weighted Gene Co-Expression Network Analysis (WGCNA) algorithm, said co-expression relationship is determined from said expression profiles or both.

5. (canceled)

6. The method of claim 1, wherein said receptors known to be associated with said biological process have been experimentally confirmed to be associated with said biological process, said dataset comprises expression profiles for all genes expressed in said target tissue, or both.

7. The method of claim 1, wherein said receptors known to not be associated with said biological process are determined using a Positive unlabeled (PU) support vector machines (SVM) bagging algorithm.

8. (canceled)

9. The method of claim 1, wherein said pathways enrichment analysis comprises KEGG pathway analysis.

10. The method of claim 1, wherein said enrichment score is an enrichment score for an entire cluster and not for an individual gene.

11. The method of claim 1, wherein said machine learning model is selected from a SVM classifier and a k-nearest neighbor (k-NN) classifier.

12. (canceled)

13. The method of claim 1, further comprising:
at an inference stage, receiving, as input, a receptor absent from said annotated training set, and enrichment scores for pathways assigned to a cluster of genes containing a gene encoding said receptor absent from said annotated training set, wherein said cluster of genes containing said gene encoding said receptor absent from said annotated training set is generated by co-expression network analysis of expression profiles of genes in said target tissue; and
applying said trained machine learning model to said input to identify a receptor associated with said biological process in said target tissue.

14. The method of claim 13, wherein said receptor absent from said annotated training set is a receptor of unknown association with said biological process in said target tissue or is a receptor absent from said first list and said second list.

15. (canceled)

16. The method of claim 13, wherein said enrichment scores for pathways assigned to a cluster of genes containing said gene that encodes said receptor absent from said annotated training set are generated by a method comprising: receiving a dataset comprising expression profiles in said target tissue for genes encoding proteins, wherein said proteins include said receptor absent from said annotated training set, applying, to said dataset, co-expression network analysis to group said genes into clusters, based on a co-expression relationship between said genes in said target tissue; and applying to said clusters, a pathways enrichment analysis to assign enrichment scores to said clusters for each pathway of said enrichment analysis, wherein said each pathway is a pathway of a specific biological process.

17. A method comprising:
receiving, as input, a dataset comprising gene expression profiles with respect to a plurality of genes associated with a corresponding plurality of tissues, wherein at least one of said genes encode a receptor;
applying, to said dataset, gene co-expression network analysis to group said genes into tissue-specific clusters, based on a co-expression relationship between said genes in tissues of said plurality of tissues;
applying, to said tissue-specific clusters, a pathways enrichment analysis to assign an enrichment score to said tissue-specific clusters, wherein at least one of said pathways is a pathway of a specific biological process; and
identifying a gene encoding a receptor included in more than one cluster having an enrichment score for a pathway of said specific biological process above a predetermined threshold as a receptor which is associated with said specified biological process.

18. The method of claim 17, wherein said receptor is selected from a cell surface receptors and an internal receptor within a cell, said expression profiles are mRNA expression profiles or both.

19. (canceled)

20. The method of claim 17 to 19, wherein said gene co-expression network analysis comprises employing a Weighted Gene Co-Expression Network Analysis (WGCNA) algorithm.

21. The method of claim 17, wherein said identifying comprises at least one of:
a. identifying genes encoding receptors included in at least five clusters having an enrichment score for a pathway of said specific biological process above a predetermined threshold;
b. identifying genes encoding receptors with correlation to an eigengene of said cluster above a predetermined threshold; and
c. applying a machine learning model trained on receptors associated with said biological process and receptors not associated with said biological process.

22. (canceled)

23. (canceled)

24. The method of claim 21, wherein said machine learning model is trained by a method comprising:
receiving a first list of receptors known to be associated with said biological process and expressed in said target tissue and a second list of receptors known to be expressed in said target tissue and not associated with said biological process;
receiving a dataset comprising expression profiles in said target tissue for genes encoding proteins, wherein said proteins include receptors of said first list and receptors of said second list;
applying, to said dataset, gene co-expression network analysis to group said genes into clusters, based on a co-expression relationship between said genes in said target tissue;
applying to said clusters, a pathways enrichment analysis to assign enrichment scores to said clusters for each pathway of said enrichment analysis, wherein said each pathway is a pathway of a specific biological process;
labeling receptors from said first list and receptors from said second list with labels comprising said enrichment score for said each pathway assigned to a cluster containing said gene encoding said receptor;
generating an annotated training set comprising receptors from said first list and receptors from said second list and corresponding labels; and
training said machine learning model on said annotated training set to produce a trained machine learning model.

25. A method of determining if a receptor is associated with a biological process in a target tissue, the method comprising:
receiving, as input, a receptor of unkonwn association with said biological process in said target tissue, and enrichment scores for pathways assigned to a cluster of genes containing a gene encoding said receptor of unknown association, wherein said cluster is generated by gene co-expression network analysis of expression profiles in said target tissue of a set of genes which includes said gene encoding said receptor of unknown association; and
applying a trained machine learning model to said input to determine if said receptor of unknown association is associated with said biological process in said target tissue, wherein said trained machine learning model has been trained on a training set comprising a first list of receptors known to be associated with said biological process and expressed in said target tissue labeled with labels comprising enrichment scores for pathways assigned to a cluster of genes containing a gene encoding said receptors of said first list, and a second list of receptors known to be expressed in said target tissue and not associated with said biological process labeled with labels comprising enrichment scores for pathways assigned to a cluster of genes containing a gene encoding receptors of said second list;
thereby determining if a receptor is associated with a biological process in a target tissue.

26. The method of claim 25, wherein at least one of:
a. said receptors are cell surface receptors, internal receptors within a cell or a combination thereof;
b. said expression profiles are mRNA expression profiles;
c. said gene co-expression network analysis comprises employing a Weighted Gene Co-Expression Network Analysis (WGCNA) algorithm
d. said receptors known to be associated with said biological process have been experimentally confirmed to be associated with said biological process;
e. said receptors known to not be associated with said biological process are determined using a Positive unlabeled (PU) support vector machines (SVM) bagging algorithm;
f. said pathways enrichment analysis comprises KEGG pathway analysis;
g. said enrichment score is an enrichment score for an entire cluster and not for an individual gene; and
h. said machine learning model is selected from a SVM classifier and a k-nearest neighbor (k-NN) classifier.

27. (canceled)

28. (canceled)

29. (canceled)

30. (canceled)

31. (canceled)

32. (canceled)

33. (canceled)

34. (canceled)

35. A system comprising:
at least one hardware processor; and
a non-transitory computer-readable storage medium having stored thereon program code, the program code executable by the at least one hardware processor to perform a method of claim 1.

36. A computer program product comprising a non-transitory computer-readable storage medium having program code embodied therewith, the program code executable by at least one hardware processor to perform a method of claim 1.
